(19) United States
(12) Patent Application Publication
(10) Pub. No.: US 2012/0046455 A1
(43) Pub. Date: Feb. 23, 2012

(54) Catalytic Cyclopropanation of Alkenes with Alpha-Cyano-Diazoacetates

(76) Inventors: X. Peter Zhang, Tampa, FL (US); Shifa Zhu, Tampa, FL (US)

(21) Appl. No.: 13/211,097

(22) Filed: Aug. 16, 2011

Related U.S. Application Data
(60) Provisional application No. 61/374,486, filed on Aug. 17, 2010.

(51) Int. Cl. C07C 253/10; C07C 245/14; C07C 255/46

(52) U.S. Cl. 534/558; 558/434; 558/426; 558/429

(57) ABSTRACT

A process for the preparation of a 1,1-cyclopropane(nitrile)(electron-acceptor), the process comprising treating an olefin with an acceptor/acceptor-substituted α-cyanodiazo reagent in the presence of a catalytic amount of a metal porphyrin catalyst.

### CROSS-REFERENCE TO RELATED APPLICATIONS

The present application claims the benefit of U.S. Provisional Application Ser. No. 61/374,486, filed Aug. 17, 2010, which is hereby incorporated by reference herein in its entirety.

### STATEMENT OF GOVERNMENT RIGHTS

This invention was made with Government support under grant number NSF #0711024, awarded by the National Science Foundation, Division of Chemistry. The Government has certain rights in the invention.

### BACKGROUND OF THE INVENTION

Catalytic asymmetric cyclopropanation of alkenes with α-diazo reagents represents one of the most general and direct approaches for stereoselective synthesis of chiral cyclopropane derivatives, which have found widespread applications. See, e.g., Carson et al., Chem. Soc. Rev. 2009, 38, 3051; Reichelt et al., Acc. Chem. Res. 2006, 39, 433; Yu et al., Tetrahedron 2005, 61, 321; and Reissig et al., Chem. Rev. 2003, 103, 1151. Considering the common existence of alkene units and the broad accessibility of diazo reagents with various combinations of α-groups, this thermodynamically favorable approach should, in principle, permit the construction of three-membered, all-carbon ring structures bearing all types of substituted functionalities. During the past two decades, a number of highly effective catalytic systems have been developed for various asymmetric cyclopropanation reactions with donor/acceptor-as well as acceptor-substituted diazo reagents. The potential of catalytic asymmetric cyclopropanation, however, has not been fully extended to acceptor/acceptor-substituted diazo reagents owing to their low reactivity and poor stereocontrollability. See, e.g., (a) Pellissier, H. Tetrahedron 2008, 64, 7041. (b) Lebel et al., Chem. Rev. 2003, 103, 977; (c) Davies et al., Org. React. 2001, 57, 1. (e) Doyle et al., Chem. Rev. 1998, 98, 911; (a) Doyle, Agnew. Chem. Int. Ed. 2009, 48, 850. (b) Doyle, J. Org. Chem. 2006, 71, 9253; and (c) Davies et al., J. Chem. Rev. 2003, 103, 2861. For select examples of asymmetric cyclopropanation with acceptor/acceptor diazo reagents, see: (a) Doyle et al., Org. Lett. 2000, 2, 1145; (b) Charette et al., J. Mol. Catal. A 2003, 196, 83; (c) Muller et al., Tetrahedron 2004, 60, 4755; (d) Zhu et al., Agnew. Chem. Int. Ed. 2008, 47, 8460; and (e) Marcoux et al., Agnew. Chem. Int. Ed. 2008, 47, 10155.

α-Cyanodiazoacetates (CDA) are a class of known acceptor/acceptor-substituted diazo reagents that have not been successfully employed for asymmetric olefin cyclopropanation. For asymmetric cyclopropanation with diazoacetonitrile, see: (a) Felpin et al., J. Org. Chem. 2001, 66, 305. and (b) Ferrand et al., Tetrahedron: Asymmetry 2005, 16, 3829. For asymmetric cyclopropanation with α-phenyldiazoacetonitrile, see: Denton et al., Chem. Commun. 2008, 1238. For asymmetric cyclopropanation with α-cyanodiazoacetamides, see: Marcoux et al., J. Am. Chem. Soc. 2009, 131, 6970. For asymmetric intramolecular cyclopropanation with allylic α-cyanodiazoacetates, see: Lin et al., Adv. Synth. Catal. 2005, 347, 1547. This catalytic process would be highly desirable, as the resulting cyclopropanes bearing geminal nitrile and ester functionalities can be further transformed into a number of densely functionalized chiral cyclopropane molecules, including synthetically and biologically important cyclopropyl-amino acids and amino alcohols as illustrated in Reaction Scheme A1 (synthesis of 1,1-cyclopropanenitrileesters via catalytic asymmetric cyclopropanation and their further transformations). See, for example, (a) Brackmann et al., Chem. Rev. 2007, 107, 4493. (b) Gnad et al., Chem. Rev. 2003, 103, 1603. (c) Fulop, Chem. Rev. 2001, 101, 2181. (d) Schwarz et al., J. Med. Chem. 2005, 48, 3026. While the only study on the intramolecular version of the catalytic process gave results that are highly substrate-dependent (yield: 11-85%; ee: 29-91%), it is strikingly noted that there has been no prior report on intermolecular asymmetric cyclopropanation with CDA. Moreover, the only intermolecular report of the catalytic process is non-asymmetric with poor diastereoselectivity (10-20% de), which highlights the challenges of stereocontrol for cyclopropanation with acceptor/acceptor-substituted diazo reagents. It is evident that this important catalytic cyclopropanation process is largely undeveloped, and that more reactive and stereodiscriminating catalysts are needed to address this unsolved problem in the field.

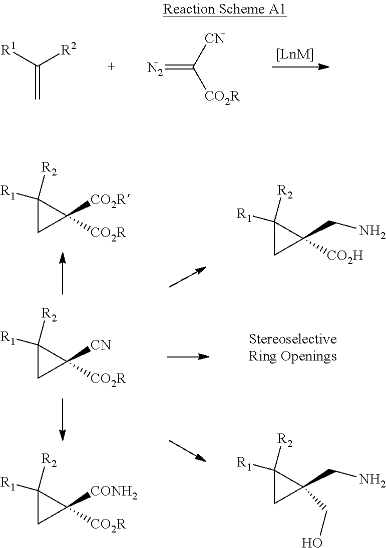

Structurally well-defined cobalt(II) complexes of D₂-symmetric chiral porphyrins [Co(D₂-Por*)] have emerged as a new class of effective catalysts for asymmetric cyclopropanation. See, e.g., Doyle, Agnew. Chem. Int. Ed. 2009, 48, 850. For selected examples of Co(II)-catalyzed asymmetric cyclopropanation, see: (a) Nakamura et al. J. Am. Chem. Soc. 1978, 100, 3443. (b) Ikeno et al., Bull. Chem. Soc. Jpn. 2001, 74, 2139. (c) Niimi et al., Adv. Synth. Catal. 2001, 343, 79. (d) Chen et al., J. Am. Chem. Soc. 2004, 126, 14718. (e) Chen et al., J. Am. Chem. Soc. 2007, 129, 12074. (f) Zhu et al., J. Am. Chem. Soc. 2008, 130, 5042. (g) Fantauzzi et al., Organometallics 2008, 27, 6143. The Co(II)-based metalloradical cyclopropanation (MRC) has been shown to possess a different reactivity profile from the widely-studied Rh₂- and Cu-based systems and can achieve highly stereoselective cyclopropanation of electron-deficient olefins and with acceptor/acceptor-substituted diazo reagents. See, e.g., Zhu et al., Agnew. Chem. Int. Ed. 2008, 47, 8460. In addition to the wide substrate scope and high selectivity (both diastereo- and enantioselectivity), the Co(II)-based MRC (metalloradical cyclopropanation) catalytic system enjoys a practical attribute that is atypical for metal-catalyzed carbene transfers: it can be operated in a one-pot fashion with alkenes as limiting reagents and requires no slow addition of diazo reagents.

Regarding the family of [Co(D2-Por*)] with tunable electronic, steric and chiral environments, our previous study revealed that [Co(P1)], the cobalt(II) complex of the D₂-symmetric chiral porphyrin 3,5-DitBu-ChenPhyrin (FIG. 1: A), is the optimal catalyst for asymmetric olefin cyclopropanation with α-nitrodiazoacetates (NDA). See, e.g., Doyle, Agnew. Chem. Int. Ed. 2009, 48, 850; Chen et al., J. Am. Chem. Soc. 2004, 126, 14718; Fantauzzi et al., Organometallics 2008, 27, 6143; and Zhu et al., Agnew. Chem. Int. Ed. 2008, 47, 8460.

### SUMMARY OF THE INVENTION

We rationalized the catalytic effectiveness of [Co(P1)] toward NDA as a consequence of two potential N—H—O hydrogen bonding interactions between two of the chiral cyclopropyl amide N—H elements on the P1 ligand with both the N═O (—NO₂ group) and the C═O (—CO₂Et group) units of the carbene moiety, respectively, in a postulated metallocarbene intermediate. Given that a cyano group is normally considered a stronger hydrogen bond acceptor than a nitro group, we envisioned a similar cobalt-carbene intermediate with the unique double-hydrogen bonding to be also potentially operative for CDA reactions (FIG. 1: B). See, e.g., (a) Laurence et al., Perspect. Drug Discovery Des. 2000, 18, 39. (b) Le Questel et al., J. Phys. Org. Chem. 2000, 13, 347. On the basis of this hypothesis, initial efforts were made to systematically investigate asymmetric cyclopropanation reactions of styrene as a model substrate with CDA by [Co(P1)] under different conditions.

In view of their unique catalytic activities, we have investigated the potential of [Co(D₂-Por*)]-based catalysts for asymmetric cyclopropanation with α-cyano diazoacetates and describe herein a catalytic system that is efficient for stereoselective cyclopropanation with α-cyano diazoacetates. In addition to high yields, the Co(II)-catalyzed reactions allow for excellent control of both diastereo- and enantioselectivity. Furthermore, the catalytic process has a broad substrate scope and can cyclopropanate both aromatic and aliphatic olefins having a wide range of electronic properties.

Among the various aspects of the present invention may be noted a process for the preparation of a 1,1-cyclopropane(nitrile)(electron-acceptor). The process comprising treating an olefin with an acceptor/acceptor-substituted α-cyanodiazo reagent in the presence of a catalytic amount of a porphyrin catalyst. In a preferred embodiment, the porporphyrin catalyst is a D₂-symmetric chiral porphyrin.

Another aspect of the present invention is a process for the preparation of a 1,1-cyclopropane(nitrile)(electron-acceptor) correponding to Formula CP comprising treating an olefin corresponding to Formula O-1 with a diazo reagent corresponding to Formula D-1 in the presence of a D₂-symmetric chiral porphyrin

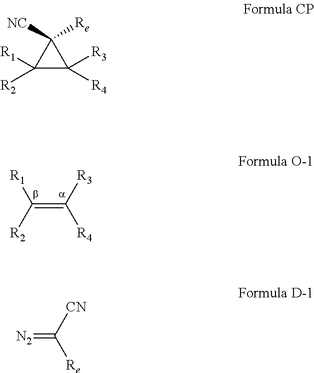

wherein Rₑ is an electron-acceptor and R₁, R₂, R₃, and R₄ are independently hydrogen, hydrocarbyl, substituted hydrocarbyl, heterocyclo, or an electron withdrawing group.

Another aspect of the present invention is a 1,1-cyclopropane(nitrile)(electron-acceptor) corresponding to Formula CP:

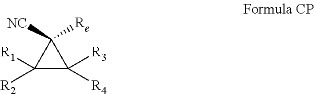

wherein R₁, R₂, R₃, and R₄ are independently hydrogen, hydrocarbyl, substituted hydrocarbyl, heterocyclo, or an electron withdrawing group, and Rₑ is an electron-acceptor.

Another aspect of the present invention is a diazo reagent corresponding to Formula D-1

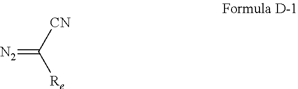

wherein Rₑ is an electron-acceptor.

Other objects and features will be in part apparent and in part pointed out hereinafter.

### BRIEF DESCRIPTION OF THE DRAWINGS

FIG. 1 depicts the chemical structure of two Co(II) porphyrins. FIG. 1A depicts a Co(II) porphyrin designated [Co(P1)] wherein P1=3,5-DitBu-ChenPhyrin. FIG. 1B depicts potential double H-bonding interaction in postulated carbene complex of [Co(P1)].

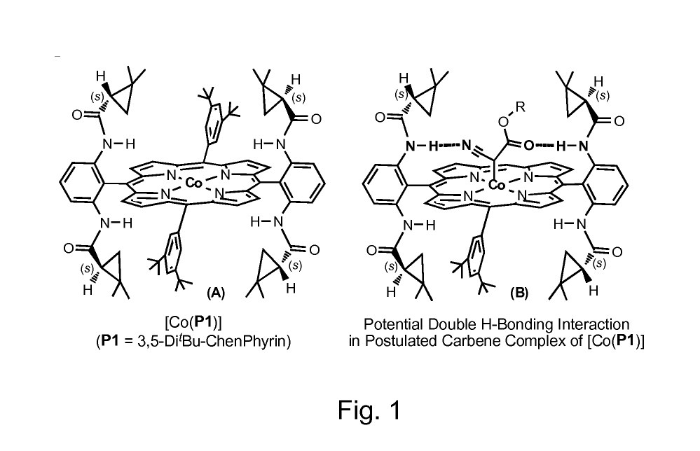

### ABBREVIATIONS AND DEFINITIONS

The following definitions and methods are provided to better define the present invention and to guide those of ordinary skill in the art in the practice of the present invention. Unless otherwise noted, terms are to be understood according to conventional usage by those of ordinary skill in the relevant art.

The term “acyl,” as used herein alone or as part of another group, denotes the moiety formed by removal of the hydroxyl group from the group —COOH of an organic carboxylic acid, e.g., RC(O)—, wherein R is R¹, R¹O—, R¹R²N— or R¹S—, R¹ is hydrocarbyl, heterosubstituted hydrocarbyl, or heterocyclo and R² is hydrogen, hydrocarbyl or substituted hydrocarbyl.

The term “acyloxy,” as used herein alone or as part of another group, denotes an acyl group as described above bonded through an oxygen linkage (—O—), e.g., RC(O)O— wherein R is as defined in connection with the term “acyl.”

Unless otherwise indicated, the alkenyl groups described herein are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

The terms “alkoxy” or “alkoxyl” as used herein alone or as part of another group denote any univalent radical, RO— where R is an alkyl group.

Unless otherwise indicated, the alkyl groups described herein are preferably lower alkyl containing from one to eight carbon atoms in the principal chain and up to 20 carbon atoms. They may be straight or branched chain or cyclic and include methyl, ethyl, propyl, isopropyl, butyl, hexyl, and the like. The substituted alkyl groups described herein may have, as substituents, any of the substituents identified as substituted hydrocarbyl substituents.

Unless otherwise indicated, the alkynyl groups described herein are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The terms “aryl” or “ar” as used herein alone or as part of another group denote optionally substituted homocyclic aromatic groups, preferably monocyclic or bicyclic groups containing from 6 to 12 carbons in the ring portion, as phenyl, biphenyl, naphthyl, substituted phenyl, substituted biphenyl or substituted naphthyl. Phenyl and substituted phenyl are the more preferred aryl. The substituted aryl groups described herein may have, as substituents, any of the substituents identified as substituted hydrocarbyl substituents.

The terms “diazo” or “azo” as used herein alone or as part of another group denote an organic compound with two linked nitrogen compounds. These moieties include without limitation diazomethane, ethyl diazoacetate, and t-butyl diazoacetate.

The term “electron acceptor” as used herein denotes a chemical moiety that accepts electrons. Stated differently, an electron acceptor is a chemical moiety that accepts either a fractional electronic charge from an electron donor moiety to form a charge transfer complex, accepts one electron from an electron donor moiety in a reduction-oxidation reaction, or accepts a paired set of electrons from an electron donor moiety to form a covalent bond with the electron donor moiety.

The terms “halogen” or “halo” as used herein alone or as part of another group denote chlorine, bromine, fluorine, and iodine.

The term “heteroaromatic” as used herein alone or as part of another group denotes optionally substituted aromatic groups having at least one heteroatom in at least one ring, and preferably 5 or 6 atoms in each ring. The heteroaromatic group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heteroaromatics include furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxyl, protected hydroxyl, acyl, acyloxy, alkoxy, alkenoxy, alkynoxy, aryloxy, halogen, amido, amino, nitro, cyano, thiol, ketals, acetals, esters and ethers.

The term “heteroatom” as used herein denotes atoms other than carbon and hydrogen.

The terms “heterocyclo” or “heterocyclic” as used herein alone or as part of another group denote optionally substituted, fully saturated or unsaturated, monocyclic or bicyclic, aromatic or nonaromatic groups having at least one heteroatom in at least one ring, and preferably 5 or 6 atoms in each ring. The heterocyclo group preferably has 1 or 2 oxygen atoms, 1 or 2 sulfur atoms, and/or 1 to 4 nitrogen atoms in the ring, and may be bonded to the remainded of the molecule through a carbon or heteroatom. Exemplary heterocyclo include heteroaromatics as furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxyl, protected hydroxyl, acyl, acyloxy, alkoxy, alkenoxy, alkynoxy, aryloxy, halogen, amido, amino, nitro, cyano, thiol, ketals, acetals, esters and ethers.

The terms “hydrocarbon” and “hydrocarbyl” as used herein alone or as part of another group denote organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, as alkaryl, alkenaryl, and alkynaryl. Unless otherwise indicated, these moieties preferably comprise 1 to 20 carbon atoms.

The term porphyrin refers to a compound comprising a fundamental skeleton of four pyrrole nuclei united through the α-positions by four methane groups to form the following macrocyclic structure:

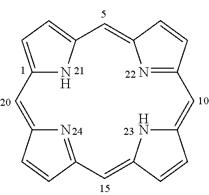

The “substituted hydrocarbyl” moieties described herein, e.g., the substituted alkyl, the substituted alkenyl, the substituted alkynyl, and the substituted aryl moieties, are hydrocarbyl moieties which are substituted with at least one atom other than carbon, including moieties in which a carbon chain atom is substituted with a hetero atom such as nitrogen, oxygen, silicon, phosphorous, boron, sulfur, or a halogen atom. These substitutents include halogen, heterocyclo, alkoxy, alkenoxy, alkynoxy, aryloxy, hydroxyl, protected hydroxy, keto, acyl, acyloxy, nitro, amino, amido, nitro, cyano, thiol, ketals, acetals, esters and ethers.

### DETAILED DESCRIPTION OF THE PREFERRED EMBODIMENTS

In accordance with the process of the present invention, compounds containing an ethylenic bond, commonly known as olefins, are cyclopropanated with a diazo reagent in the presence of a cobalt porphyrin complex. Advantageously, the diazo reagent is an acceptor/acceptor-substituted diazo reagent corresponding to Formula D-1

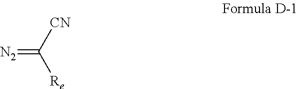

wherein Rₑ is an electron-acceptor. Exemplary electron-acceptors include hydroxy, alkoxy, mercapto, halogens, carbonyls, sulfonyls, nitrile, quaternary amines, nitro, trihalomethyl, imine, amidine, oxime, thioketone, thioester, or thioamide. In one embodiment, the electron-acceptor is hydroxy, alkoxy, mercapto, halogen, carbonyl, sulfonyl, nitrile, quaternary amine, nitro, or trihalomethyl. In another embodiment, the electron-acceptor is halogen, carbonyl, nitrile, quaternary amine, nitro, or trihalomethyl. In another embodiment, the electron-acceptor is halogen, carbonyl, nitrile, nitro, or trihalomethyl. When the electron-acceptor is alkoxy, it generally corresponds to the formula —OR where R is hydrocarbyl, substituted hydrocarbyl, or heterocyclo. When the electron-acceptor is mercapto, it generally corresponds to the formula —SR where R is hydrogen, hydrocarbyl, substituted hydrocarbyl or heterocyclo. When the electron-acceptor is a halogen atom, the electron-acceptor may be fluoro, chloro, bromo, or iodo; typically, when it is halo it will be fluoro or chloro. When the electron-acceptor is a carbonyl, it may be an aldehyde (—C(O)H), ketone (—C(O)R), ester (—C(O)OR), acid (—C(O)OH), acid halide (—C(O)X), amide (—C(O)NRₐR_{b}), or anhydride (—C(O)OC(O)R) where R is hydrocarbyl, substituted hydrocarbyl or heterocyclo, Rₐ and R_{b} are independently hydrogen, hydrocarbyl, substituted hydrocarbyl or heterocyclo, and X is a halogen atom. When the electron-acceptor is a sulfonyl, it may be an acid (—SO₃H) or a derivative thereof (—SO₂R) where R is hydrocarbyl, substituted hydrocarbyl or heterocyclo. When the electron-acceptor is a quaternary amine, it generally corresponds to the formula —N⁺RₐR_{b}R_{c} where Rₐ, R_{b} and R_{c} are independently hydrogen, hydrocarbyl, substituted hydrocarbyl or heterocyclo. When the electron-acceptor is a trihalomethyl, it is preferably trifluoromethyl or trichloromethyl. In each of the foregoing exemplary electron-acceptors containing the variable “X”, in one embodiment, X may be chloro or fluoro, preferably fluoro. In each of the foregoing exemplary electron-acceptors containing the variable “R”, R may be alkyl. In each of the foregoing exemplary electron-acceptors containing the variable “Rₐ”, “R_{b}”, and “R_{c}”, Rₐ, R_{b} and R_{c} may independently be hydrogen or alkylmetal porphyrin catalyzed process proceeds relatively efficiently under relatively mild and neutral conditions, in a one-pot fashion, with olefins as limiting reagents and without the need for slow-addition of diazo reagents.

In accordance with one preferred embodiment, the diazo reagent corresponds to Formula D-1, and the electron acceptor, Rₑ, is a halide, aldehyde, ketone, ester, carboxylic acid, amide, acyl chloride, trifluoromethyl, nitrile, sulfonic acid, ammonia, or an amine. In this embodiment, for example, the electron-acceptor, Rₑ, may be selected from the group consisting of —X, —C(O)H, —C(O)R, —C(O)OR, —C(O)OH, —C(O)X, —C(X)₃, —CN, —SO₃H, —N⁺H₃, and —N⁺(R)₃ where R is hydrocarbyl, substituted hydrocarbyl or heterocyclo and X is halogen. In one such embodiment, the electron-acceptor, Rₑ, is selected from the group consisting of —C(O)H, —C(O)R, —C(O)OR, —C(O)OH, —C(O)X, —CN, —SO₃H, —N⁺H₃, and —N⁺(R)₃ where R is hydrocarbyl, substituted hydrocarbyl or heterocyclo and X is halogen.

In one preferred embodiment, the diazo reagent has the formula N₂C(CN)C(O)OR₁₀ where R₁₀ is hydrocarbyl, substituted hydrocarbyl or heterocyclo. For example, in one such embodiment, the diazo reagent has the formula N₂C(CN)C(O)OR₁₀ where R₁₀ is alkyl, aryl or alkaryl. By way of further example, in one such embodiment, the diazo reagent has the formula N₂C(CN)C(O)OR₁₀ where R₁₀ is lower alkyl or aryl. Other exemplary α-cyanodiazoacetates have the formula N₂C(CN)C(O)OR₁₀ where R₁₀ is lower alkyl such as methyl, ethyl, propyl or butyl.

In one embodiment, the diazo reagent is used in the preparation of 1,1-cyclopropane(nitrile)(electron-acceptor) as depicted in Reaction Scheme 1

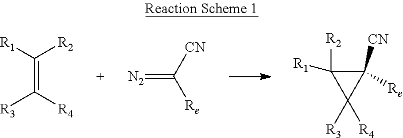

wherein R₁, R₂, R₃ and R₄ are independently hydrogen, hydrocarbyl, substituted hydrocarbyl, heterocyclo, or EWG, each EWG is independently an electron withdrawing group, R^{a} and R^{b} are independently hydrogen, hydrocarbyl, substituted hydrocarbyl, or heterocyclo, and Rₑ is as defined in connection with Formula D-1. For example, in one such embodiment, the diazo reagent has the formula, N₂C(CN)C(O)OR₁₀, where R₁₀ is hydrocarbyl, substituted hydrocarbyl or heterocyclo. By way of further example, in one such embodiment, the diazo reagent has the formula N₂C(CN)C(O)OR₁₀ where R₁₀ is alkyl, aryl or alkaryl, more lower alkyl or aryl. The reaction is preferably carried out in the presence of a catalytic amount of a cobalt porphyrin catalyst.

Olefin Substrates

In general, olefins may be used as substrates to form a 1,1-cyclopropane(nitrile)(electron-acceptor). In one embodiment, the olefin corresponds to Formula O-1:

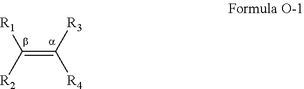

wherein R₁, R₂, R₃, and R₄ are independently hydrogen, hydrocarbyl, substituted hydrocarbyl, heterocyclo, or an electron withdrawing group. For example, in one embodiment, R₁ may be hydrogen. By way of further example, R₁ may be alkyl or substituted alkyl. By way of further example, R₁ may be aryl or substituted aryl. In one embodiment, R₂ is hydrogen. In another embodiment, R₂ is alkyl or substituted alkyl. In yet another embodiment, R₂ is aryl or substituted aryl; for example, R₂ may be phenyl or substituted phenyl wherein the phenyl substituents are selected from the group consisting of alkyl, alkoxy, halo, trihalomethyl, acyloxy, and nitro. In one embodiment, R₂ is acyl. For example, R₂ may be —C(O)R, —C(O)OR, or —C(O)NRₐR_{b} wherein R, Rₐ and R_{b} are independently optionally substituted alkyl or optionally substituted aryl. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment, R₁ and R₂ are both hydrogen. In one embodiment, one of R₁, R₂, R₃, and R₄ is an electron withdrawing group. In another embodiment, none of R₁, R₂, R₃, and R₄ is an electron withdrawing group. In one embodiment, R₃, R₄ and the α-carbon, or R₁, R₂ and the β-carbon form a carbocyclic or heterocyclic ring. In another embodiment, R₁, R₃, the α-carbon, and the β-carbon or R₂, R₄, the α-carbon, and the β-carbon form a carbocyclic or heterocyclic ring. In another embodiment, R₁, R₄, the α-carbon, and the β-carbon or R₂, R₃, the α-carbon, and the β-carbon form a carbocyclic or heterocyclic ring.

When the olefin corresponds to Formula I and one, but only one of R₁, R₂, R₃, and R₄ is an electron withdrawing group, e.g., R₁ is an electron withdrawing group, the olefin corresponds to Formula O-1-EWG:

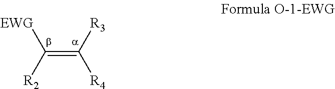

wherein EWG is an electron withdrawing group and R₂, R₃, and R₄ are as defined in connection with Formula O-1. That is, R₂, R₃, and R₄ are independently hydrogen, hydrocarbyl, substituted hydrocarbyl, heterocyclo, or an electron withdrawing group. In one embodiment, R₂ is hydrogen. In another embodiment, R₂ is alkyl or substituted alkyl. In another embodiment, R₂ is aryl or substituted aryl; for example, R₂ may be phenyl or substituted phenyl wherein the phenyl substituents are selected from the group consisting of alkyl, alkoxy, halo, trihalomethyl, acyloxy, and nitro. In one embodiment, R₂ is acyl. For example, in one embodiment, R₂ is —C(O)R, —C(O)OR, or —C(O)NRₐR_{b} wherein R, Rₐ and R_{b} are independently optionally substituted alkyl or optionally substituted aryl. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment, one of R₂, R₃, and R₄ is an electron withdrawing group. In one embodiment, R₃, R₄ and the α-carbon form a carbocyclic or heterocyclic ring. In another embodiment, R₂, R₄, the α-carbon, and the β-carbon form a carbocyclic or heterocyclic ring. In another embodiment, R₂, R₃, the α-carbon, and the β-carbon form a carbocyclic or heterocyclic ring.

When the olefin corresponds to Formula O-1 and R₁ is hydrogen, the olefin corresponds to Formula O-2:

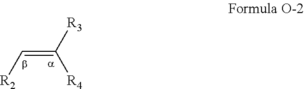

wherein R₂, R₃, and R₄ are independently hydrogen, hydrocarbyl, substituted hydrocarbyl, heterocyclo, or an electron withdrawing group. In one embodiment, R₂ is hydrogen. In another embodiment, R₂ is alkyl or substituted alkyl. In another embodiment, R₂ is aryl or substituted aryl; for example, R₂ may be phenyl or substituted phenyl wherein the phenyl substituents are selected from the group consisting of alkyl, alkoxy, halo, trihalomethyl, acyloxy, and nitro. In one embodiment, R₂ is acyl; for example, R₂ may be —C(O)R, —C(O)OR, or —C(O)NRₐR_{b} wherein R, Rₐ and R_{b} are independently optionally substituted alkyl or optionally substituted aryl. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment, R₃ and R₄ are both hydrogen. In one embodiment, one of R₂, R₃, and R₄ is an electron withdrawing group. In one embodiment, R₂ is an electron withdrawing group selected from the group consisting of X, —C(O)H, —C(O)R, —C(O)OR, —C(O)OH, —C(O)X, —C(X)₃, —CN, —SO₃H, —N⁺H₃, —N⁺R₃, and —N⁺O₂ where R is hydrocarbyl, substituted hydrocarbyl or heterocyclo and X is halogen, optionally substituted alkyl or optionally substituted aryl. In another embodiment, none of R₂, R₃, and R₄ is an electron withdrawing group. In one embodiment, R₃, R₄ and the α-carbon form a carbocyclic or heterocyclic ring. In another embodiment, R₂, R₄, the α-carbon, and the β-carbon form a carbocyclic or heterocyclic ring. In another embodiment, R₂, R₃, the α-carbon, and the β-carbon form a carbocyclic or heterocyclic ring.

When the olefin corresponds to Formula O-1, R₁ is hydrogen, and one but only one of R₃ and R₄ is hydrogen, the olefin corresponds to Formula O-3-cis or Formula O-3-trans:

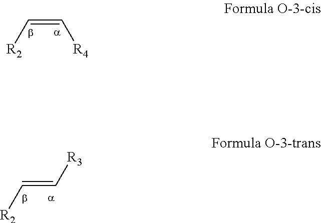

wherein R₂, R₃ and R₄ are independently hydrogen, hydrocarbyl, substituted hydrocarbyl, heterocyclo, or an electron withdrawing group. In one embodiment, R₂ is hydrogen. In another embodiment, R₂ is alkyl or substituted alkyl. In another embodiment, R₂ is aryl or substituted aryl; for example, R₂ may be phenyl or substituted phenyl wherein the phenyl substituents are selected from the group consisting of alkyl, alkoxy, halo, trihalomethyl, acyloxy, and nitro. In one embodiment, R₂ is acyl; for example, R₂ may be —C(O)R, —C(O)OR, or —C(O)NRₐR_{b} wherein R, Rₐ and R_{b} are independently optionally substituted alkyl or optionally substituted aryl. In one embodiment, R₃ is hydrogen. In another embodiment, R₃ is alkyl or substituted alkyl. In one embodiment, one of R₂ and R₃ is an electron withdrawing group. In one embodiment, R₄ is hydrogen. In another embodiment, R₄ is alkyl or substituted alkyl. In one embodiment, one of R₂ and R₄ is an electron withdrawing group. In one embodiment, R₂ is an electron withdrawing group selected from the group consisting of X, —C(O)H, —C(O)R, —C(O)OR, —C(O)OH, —C(O)X, —C(X)₃, —CN, —SO₃H, —N⁺H₃, —N⁺R₃, and —N⁺O₂ where R is hydrocarbyl, substituted hydrocarbyl or heterocyclo and X is halogen, optionally substituted alkyl or optionally substituted aryl. In one embodiment, R₂, R₄, the α-carbon, and the β-carbon form a carbocyclic or heterocyclic ring. In another embodiment, R₂, R₃, the α-carbon, and the β-carbon form a carbocyclic or heterocyclic ring.

When the olefin corresponds to Formula O-1 and R₃ and R₄ are both hydrogen, the olefin is a terminal alkene, corresponding to Formula O-4:

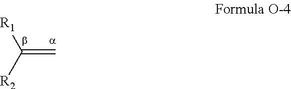

wherein R₁ and R₂ are independently hydrogen, hydrocarbyl, substituted hydrocarbyl, heterocyclo, or an electron withdrawing group. In one embodiment, one but only one of R₁ and R₂ is an electron withdrawing group. In another embodiment, neither R₁ nor R₂ is an electron withdrawing group. In another embodiment, R₁ and R₂ are independently hydrocarbyl, substituted hydrocarbyl, or heterocyclo. In one embodiment, one of R₁ and R₂ is hydrogen. In one embodiment, R₁, R₂, and the β-carbon form a carbocyclic or heterocyclic ring.

When the olefin corresponds to Formula O-1 and R₂, R₃, and R₄ are hydrogen, the olefin is a terminal olefin corresponding to Formula O-5:

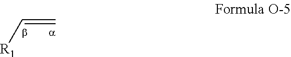

wherein R₁ is hydrogen, hydrocarbyl, substituted hydrocarbyl, heterocyclo, or an electron withdrawing group. In one embodiment, R₁ is alkyl, substituted alkyl, aryl, substituted aryl, or acyl. In one embodiment, R₁ is phenyl or substituted phenyl. In another embodiment, R₁ is alkyl. In another embodiment, R₁ is an electron withdrawing group. In one embodiment, R₂ is phenyl or substituted phenyl. In another embodiment, R₁ is acyl. In another embodiment, R₁ is R₁₁C(O)— wherein R₁₁ is alkyl, substituted alkyl, alkoxy, or amino.

In general, the olefin's electron withdrawing group(s), EWG, as depicted in Formula O-1-EWG and described in connection with Formula O-1, Formula O-2, Formula O-3-trans, Formula O-3-cis, Formula O-4 or Formula O-5, is any substituent that draws electrons away from the ethylenic bond. Exemplary electron withdrawing groups include hydroxy, alkoxy, mercapto, halogens, carbonyls, sulfonyls, nitrile, quaternary amines, nitro, trihalomethyl, imine, amidine, oxime, thioketone, thioester, or thioamide. In one embodiment, the electron withdrawing group(s) is/are hydroxy, alkoxy, mercapto, halogen, carbonyl, sulfonyl, nitrile, quaternary amine, nitro, or trihalomethyl. In another embodiment, the electron withdrawing group(s) is/are halogen, carbonyl, nitrile, quaternary amine, nitro, or trihalomethyl. In another embodiment, the electron withdrawing group(s) is/are halogen, carbonyl, nitrile, nitro, or trihalomethyl. When the electron withdrawing group is alkoxy, it generally corresponds to the formula —OR where R is hydrocarbyl, substituted hydrocarbyl, or heterocyclo. When the electron withdrawing group is mercapto, it generally corresponds to the formula —SR where R is hydrogen, hydrocarbyl, substituted hydrocarbyl or heterocyclo. When the electron withdrawing group is a halogen atom, the electron withdrawing group may be fluoro, chloro, bromo, or iodo; typically, when it is halo it will be fluoro or chloro. When the electron withdrawing group is a carbonyl, it may be an aldehyde (—C(O)H), ketone (—C(O)R), ester (—C(O)OR), acid (—C(O)OH), acid halide (—C(O)X), amide (—C(O)NEₐE_{b}), or anhydride (—C(O)OC(O)R) where R is hydrocarbyl, substituted hydrocarbyl or heterocyclo, Eₐ and E_{b} are independently hydrogen, hydrocarbyl, substituted hydrocarbyl or heterocyclo, and X is a halogen atom. When the electron withdrawing group is a sulfonyl, it may be an acid (—SO₃H) or a derivative thereof (—SO₂R) where R is hydrocarbyl, substituted hydrocarbyl or heterocyclo. When the electron withdrawing group is a quaternary amine, it generally corresponds to the formula —N⁺EₐE_{b}E_{c} where Eₐ, E_{b} and E_{c} are independently hydrogen, hydrocarbyl, substituted hydrocarbyl or heterocyclo. When the electron withdrawing group is a trihalomethyl, it is preferably trifluoromethyl or trichloromethyl. In each of the foregoing exemplary electron withdrawing groups containing the variable “X”, in one embodiment, X may be chloro or fluoro, preferably fluoro. In each of the foregoing exemplary electron withdrawing groups containing the variable “R”, R may be alkyl. In each of the foregoing exemplary electron withdrawing groups containing the variable “Eₐ”, “E_{b}”, and “E_{c},” Eₐ, E_{b} and E_{c} may independently be hydrogen or alkyl.

In accordance with one preferred embodiment, the electron withdrawing group(s) is/are a halide, aldehyde, ketone, ester, carboxylic acid, amide, acyl chloride, trifluoromethyl, nitrile, sulfonic acid, ammonia, amine, or a nitro group. In this embodiment, the electron withdrawing group(s) correspond to one of the following chemical structures: —X, —C(O)H, —C(O)R, —C(O)OR, —C(O)OH, —C(O)X, —C(X)₃, —CN, —SO₃H, —N⁺H₃, —N⁺(R)₃, or —N⁺O₂ where R is hydrocarbyl, substituted hydrocarbyl or heterocyclo and X is halogen.

As illustrated more fully in the examples, the diastereo- and enantio-selectivity can be influenced, at least in part, by selection of the metal porphyrin complex. Similarly, stereoselectivity of the reaction may also be influenced by the selection of chiral porphyrin ligands with desired electronic, steric, and chiral environments. Accordingly, the catalytic system of the present invention may advantageously be used to control stereoselectivity.

### Metal Porphyrins

The porphyrin with which the transition metal is complexed may be any of a wide range of porphyrins known in the art. Exemplary porphyrins are described in U.S. Patent Publication No. 2006/0030718 and U.S. Pat. Nos. 6,951,935 and 7,417,142 (each of which is incorporated herein by reference, in its entirety). Exemplary porphyrins are also described in Chen et al., Bromoporphyrins as Versatile Synthons for Modular Construction of Chiral Porphyrins: Cobalt-Catalyzed Highly Enantioselective and Diastereoselective Cyclopropanation (J. Am. Chem. Soc. 2004). In a preferred embodiment, the porphyrin is complexed with cobalt.

In one embodiment, the metal porphyrin complex is a cobalt(II) porphyrin complex. In one particularly preferred embodiment, the cobalt porphyrin complex is a chiral porphyrin complex corresponding to the following structure:

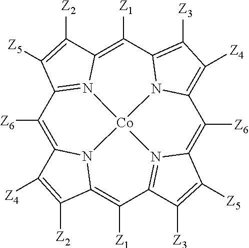

wherein each Z₁, Z₂, Z₃, Z₄, Z₅ and Z₆ are each independently selected from the group consisting of X, H, alkyl, substituted alkyls, arylalkyls, aryls and substituted aryls; and X is selected from the group consisting of halogen, trifluoromethanesulfonate (OTf), haloaryl and haloalkyl. In a preferred embodiment, Z₂, Z₃, Z₄ and Z₅ are hydrogen, Z₁ is a substituted phenyl, Z₆ is substituted phenyl, and Z₁ and Z₆ are different. In one particularly preferred embodiment, Z₂, Z₃, Z₄ and Z₅ are hydrogen, Z₁ is substituted phenyl, Z₆ is substituted phenyl, Z₁ and Z₆ are different, and the porphyrin is a chiral porphyrin. In one even further preferred embodiment, Z₂, Z₃, Z₄ and Z₅ are hydrogen, Z₁ is substituted phenyl, Z₆ is substituted phenyl, Z₁ and Z₆ are different and the porphyrin has D₂-symmetry.

In a preferred embodiment, Z₁ is selected from the group consisting of

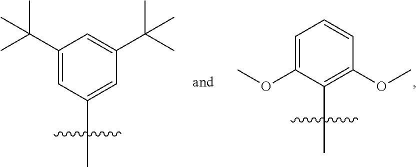

wherein

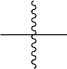

denotes the point of attachment to the porphyrin complex.

In a preferred embodiment, Z₆ is selected from the group consisting of

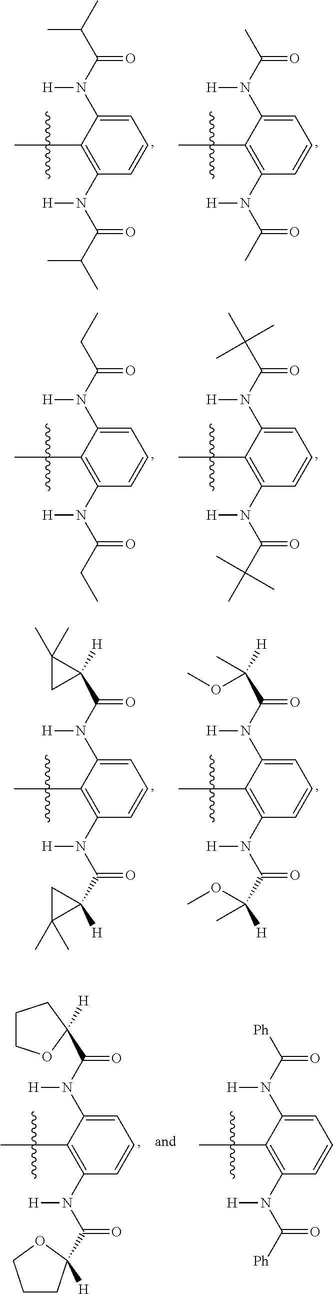

wherein

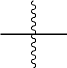

denotes the point of attachment to the porphyrin complex.

Exemplary cobalt (II) porphyrins include the following, designated [Co(P1)], [Co(P2)], [Co(P3)], [Co(P4)], [Co(P5)], and [Co(P6)]:

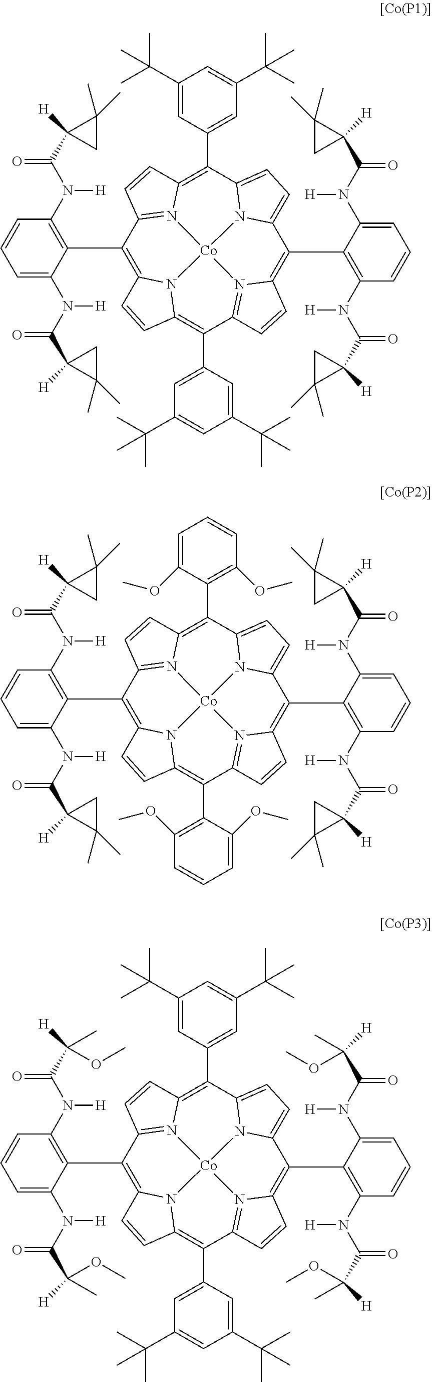

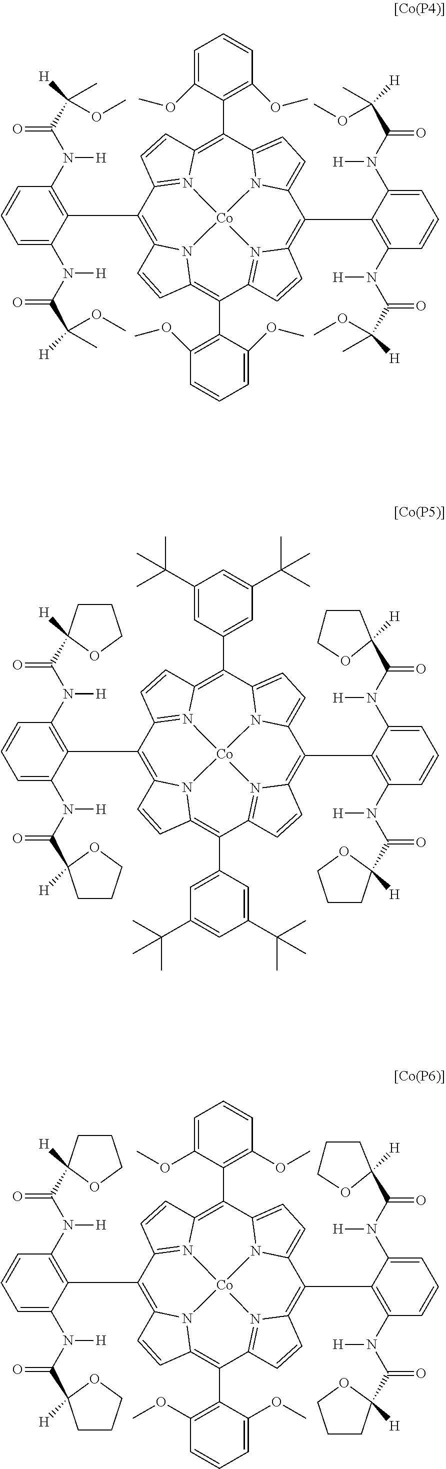

Exemplary cobalt (II) porphyrins also include the following, designated [Co(P11)], [Co(P12)], [Co(P13)], [Co(P14)], and [Co(P15)]:

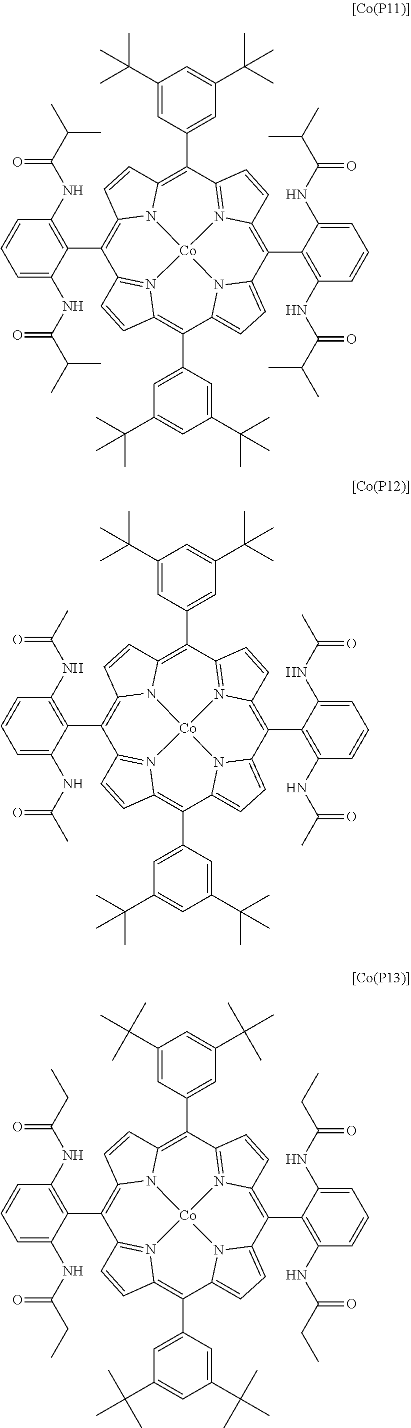

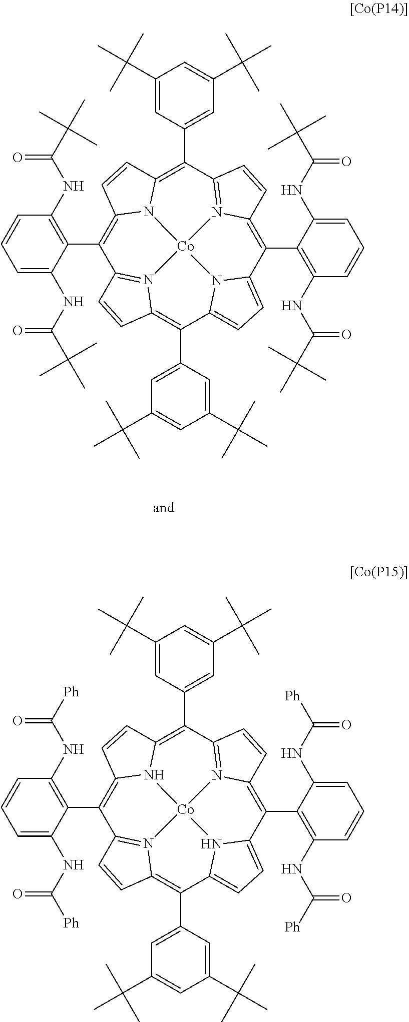

1,1-Cyclopropane(nitrile)(Electron Acceptors)

In one embodiment, the 1,1-cyclopropane(nitrile)(electron-acceptor) produced by the process of the present invention corresponds to Formula CP:

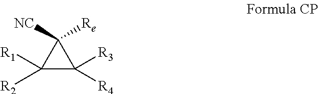

wherein R₁, R₂, R₃, and R₄ are independently hydrogen, hydrocarbyl, substituted hydrocarbyl, heterocyclo, or an electron withdrawing group, and Rₑ is an electron-acceptor. Exemplary electron-acceptors, Rₑ, include hydroxy, alkoxy, mercapto, halogens, carbonyls, sulfonyls, nitrile, quaternary amines, nitro, trihalomethyl, imine, amidine, oxime, thioketone, thioester, or thioamide. In one embodiment, the electron-acceptor, Rₑ, is hydroxy, alkoxy, mercapto, halogen, carbonyl, sulfonyl, nitrile, quaternary amine, nitro, or trihalomethyl. In another embodiment, the electron-acceptor is halogen, carbonyl, nitrile, quaternary amine, or trihalomethyl. In another embodiment, the electron-acceptor is halogen, carbonyl, nitrile, or trihalomethyl. When the electron-acceptor is alkoxy, it generally corresponds to the formula —OR where R is hydrocarbyl, substituted hydrocarbyl, or heterocyclo. When the electron-acceptor is mercapto, it generally corresponds to the formula —SR where R is hydrogen, hydrocarbyl, substituted hydrocarbyl or heterocyclo. When the electron-acceptor is a halogen atom, the electron-acceptor may be fluoro, chloro, bromo, or iodo; typically, when it is halo it will be fluoro or chloro. When the electron-acceptor is a carbonyl, it may be an aldehyde (—C(O)H), ketone (—C(O)R), ester (—C(O)OR), acid (—C(O)OH), acid halide (—C(O)X), amide (—C(O)NEₐE_{b}), or anhydride (—C(O)OC(O)R) where R is hydrocarbyl, substituted hydrocarbyl or heterocyclo, Eₐ and E_{b} are independently hydrogen, hydrocarbyl, substituted hydrocarbyl or heterocyclo, and X is a halogen atom. When the electron-acceptor is a sulfonyl, it may be an acid (—SO₃H) or a derivative thereof (—SO₂R) where R is hydrocarbyl, substituted hydrocarbyl or heterocyclo. When the electron-acceptor is a quaternary amine, it generally corresponds to the formula —N⁺EₐE_{b}E_{c} where Eₐ, E_{b} and E_{c} are independently hydrogen, hydrocarbyl, substituted hydrocarbyl or heterocyclo. When the electron-acceptor is a trihalomethyl, it is preferably trifluoromethyl or trichloromethyl. In each of the foregoing exemplary electron-acceptors containing the variable “X”, in one embodiment, X may be chloro or fluoro, preferably fluoro. In each of the foregoing exemplary electron-acceptors containing the variable “R”, R may be alkyl. In each of the foregoing exemplary electron-acceptors containing the variable “Eₐ,” “E_{b},” and “E_{c},” Eₐ, E_{b}, and E_{c} may independently be hydrogen or alkylmetal porphyrin catalyzed process proceeds relatively efficiently under relatively mild and neutral conditions, in a one-pot fashion, with olefins as limiting reagents and without the need for slow-addition of diazo reagents.

In one embodiment, the 1,1-cyclopropane(nitrile)(electron-acceptor) produced by the process of the present invention corresponds to Formula CP-1:

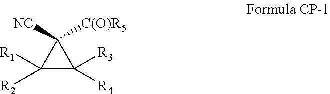

wherein R₁, R₂, R₃, and R₄ are independently hydrogen, hydrocarbyl, substituted hydrocarbyl, heterocyclo, or an electron withdrawing group, R₅ is hydrogen, hydrocarbyl, substituted hydrocarbyl, —OR₆, or —NEₐE_{b}, R₆ is hydrogen, hydrocarbyl, substituted hydrocarbyl or heterocyclo, and Eₐ and E_{b} are independently hydrogen, hydrocarbyl, substituted hydrocarbyl or heterocyclo. In one exemplary embodiment, R₅ is hydrogen. In one exemplary embodiment, R₅ is hydrocarbyl, substituted hydrocarbyl or heterocyclo. In one exemplary embodiment, R₅ is —OR₆ and R₆ is hydrocarbyl, substituted hydrocarbyl or heterocyclo. In one exemplary embodiment, R₅ is —NEₐE_{b}, and Eₐ and E_{b} are independently hydrogen, hydrocarbyl, substituted hydrocarbyl or heterocyclo.

In one embodiment, the 1,1-cyclopropane(nitrile)(electron-acceptor) produced by the process of the present invention corresponds to Formula CP-2:

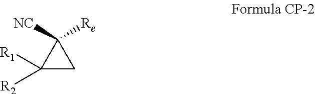

wherein R₁ and R₂ are independently hydrogen, hydrocarbyl, substituted hydrocarbyl, heterocyclo, or an electron withdrawing group, and Rₑ is an electron-acceptor. Exemplary electron-acceptors, Rₑ, include hydroxy, alkoxy, mercapto, halogens, carbonyls, sulfonyls, nitrile, quaternary amines, nitro, trihalomethyl, imine, amidine, oxime, thioketone, thioester, or thioamide. In one embodiment, the electron-acceptor, Rₑ, is hydroxy, alkoxy, mercapto, halogen, carbonyl, sulfonyl, nitrile, quaternary amine, nitro, or trihalomethyl. In another embodiment, the electron-acceptor is halogen, carbonyl, nitrile, quaternary amine, or trihalomethyl. In another embodiment, the electron-acceptor is halogen, carbonyl, nitrile, or trihalomethyl. When the electron-acceptor is alkoxy, it generally corresponds to the formula —OR where R is hydrocarbyl, substituted hydrocarbyl, or heterocyclo. When the electron-acceptor is mercapto, it generally corresponds to the formula —SR where R is hydrogen, hydrocarbyl, substituted hydrocarbyl or heterocyclo. When the electron-acceptor is a halogen atom, the electron-acceptor may be fluoro, chloro, bromo, or iodo; typically, when it is halo it will be fluoro or chloro. When the electron-acceptor is a carbonyl, it may be an aldehyde (—C(O)H), ketone (—C(O)R), ester (—C(O)OR), acid (—C(O)OH), acid halide (—C(O)X), amide (—C(O)NEₐE_{b}), or anhydride (—C(O)OC(O)R) where R is hydrocarbyl, substituted hydrocarbyl or heterocyclo, Eₐ and E_{b} are independently hydrogen, hydrocarbyl, substituted hydrocarbyl or heterocyclo, and X is a halogen atom. When the electron-acceptor is a sulfonyl, it may be an acid (—SO₃H) or a derivative thereof (—SO₂R) where R is hydrocarbyl, substituted hydrocarbyl or heterocyclo. When the electron-acceptor is a quaternary amine, it generally corresponds to the formula —N⁺EₐE_{b}E_{c} where Eₐ, E_{b} and E_{c} are independently hydrogen, hydrocarbyl, substituted hydrocarbyl or heterocyclo. When the electron-acceptor is a trihalomethyl, it is preferably trifluoromethyl or trichloromethyl. In each of the foregoing exemplary electron-acceptors containing the variable “X”, in one embodiment, X may be chloro or fluoro, preferably fluoro. In each of the foregoing exemplary electron-acceptors containing the variable “R”, R may be alkyl. In each of the foregoing exemplary electron-acceptors containing the variable “Eₐ,” “E_{b},” and “E_{c},” Eₐ, E_{b}, and E_{c} may independently be hydrogen or alkylmetal porphyrin catalyzed process proceeds relatively efficiently under relatively mild and neutral conditions, in a one-pot fashion, with olefins as limiting reagents and without the need for slow-addition of diazo reagents.

In one embodiment, the 1,1-cyclopropane(nitrile)(electron-acceptor) produced by the process of the present invention corresponds to Formula CP-3:

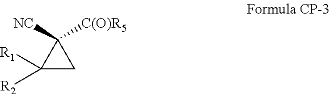

wherein R₁ and R₂ are independently hydrogen, hydrocarbyl, substituted hydrocarbyl, heterocyclo, or an electron withdrawing group, R₅ is hydrogen, hydrocarbyl, substituted hydrocarbyl, —OR₆, or —NEₐE_{b}, R₆ is hydrogen, hydrocarbyl, substituted hydrocarbyl or heterocyclo, and Eₐ and E_{b} are independently hydrogen, hydrocarbyl, substituted hydrocarbyl or heterocyclo. In one exemplary embodiment, R₅ is hydrogen. In one exemplary embodiment, R₅ is hydrocarbyl, substituted hydrocarbyl or heterocyclo. In one exemplary embodiment, R₅ is —OR₆ and R₆ is hydrocarbyl, substituted hydrocarbyl or heterocyclo. In one exemplary embodiment, R₅ is —NEₐE_{b}, and Eₐ and E_{b} are independently hydrogen, hydrocarbyl, substituted hydrocarbyl or heterocyclo.

In one embodiment, the 1,1-cyclopropane(nitrile)(electron-acceptor) produced by the process of the present invention corresponds to Formula CP-4:

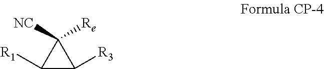

wherein R₁ and R₃ are independently hydrogen, hydrocarbyl, substituted hydrocarbyl, heterocyclo, or an electron withdrawing group, and Rₑ is an electron-acceptor. Exemplary electron-acceptors, Rₑ, include hydroxy, alkoxy, mercapto, halogens, carbonyls, sulfonyls, nitrile, quaternary amines, nitro, trihalomethyl, imine, amidine, oxime, thioketone, thioester, or thioamide. In one embodiment, the electron-acceptor, Rₑ, is hydroxy, alkoxy, mercapto, halogen, carbonyl, sulfonyl, nitrile, quaternary amine, nitro, or trihalomethyl. In another embodiment, the electron-acceptor is halogen, carbonyl, nitrile, quaternary amine, or trihalomethyl. In another embodiment, the electron-acceptor is halogen, carbonyl, nitrile, or trihalomethyl. When the electron-acceptor is alkoxy, it generally corresponds to the formula —OR where R is hydrocarbyl, substituted hydrocarbyl, or heterocyclo. When the electron-acceptor is mercapto, it generally corresponds to the formula —SR where R is hydrogen, hydrocarbyl, substituted hydrocarbyl or heterocyclo. When the electron-acceptor is a halogen atom, the electron-acceptor may be fluoro, chloro, bromo, or iodo; typically, when it is halo it will be fluoro or chloro. When the electron-acceptor is a carbonyl, it may be an aldehyde (—C(O)H), ketone (—C(O)R), ester (—C(O)OR), acid (—C(O)OH), acid halide (—C(O)X), amide (—C(O)NEₐE_{b}), or anhydride (—C(O)OC(O)R) where R is hydrocarbyl, substituted hydrocarbyl or heterocyclo, Eₐ and E_{b} are independently hydrogen, hydrocarbyl, substituted hydrocarbyl or heterocyclo, and X is a halogen atom. When the electron-acceptor is a sulfonyl, it may be an acid (—SO₃H) or a derivative thereof (—SO₂R) where R is hydrocarbyl, substituted hydrocarbyl or heterocyclo. When the electron-acceptor is a quaternary amine, it generally corresponds to the formula —N⁺EₐE_{b}E_{c} where Eₐ, E_{b} and E_{c} are independently hydrogen, hydrocarbyl, substituted hydrocarbyl or heterocyclo. When the electron-acceptor is a trihalomethyl, it is preferably trifluoromethyl or trichloromethyl. In each of the foregoing exemplary electron-acceptors containing the variable “X”, in one embodiment, X may be chloro or fluoro, preferably fluoro. In each of the foregoing exemplary electron-acceptors containing the variable “R”, R may be alkyl. In each of the foregoing exemplary electron-acceptors containing the variable “Eₐ,” “E_{b},” and “E_{c},” Eₐ, E_{b}, and E_{c} may independently be hydrogen or alkylmetal porphyrin catalyzed process proceeds relatively efficiently under relatively mild and neutral conditions, in a one-pot fashion, with olefins as limiting reagents and without the need for slow-addition of diazo reagents.

In one embodiment, the 1,1-cyclopropane(nitrile)(electron-acceptor) produced by the process of the present invention corresponds to Formula CP-5:

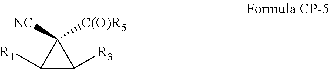

wherein R₁ and R₃ are independently hydrogen, hydrocarbyl, substituted hydrocarbyl, heterocyclo, or an electron withdrawing group, R₅ is hydrogen, hydrocarbyl, substituted hydrocarbyl, —OR₆, or —NEₐE_{b}, R₆ is hydrogen, hydrocarbyl, substituted hydrocarbyl or heterocyclo, and Eₐ and E_{b} are independently hydrogen, hydrocarbyl, substituted hydrocarbyl or heterocyclo. In one exemplary embodiment, R₅ is hydrogen. In one exemplary embodiment, R₅ is hydrocarbyl, substituted hydrocarbyl or heterocyclo. In one exemplary embodiment, R₅ is —OR₆ and R₆ is hydrocarbyl, substituted hydrocarbyl or heterocyclo. In one exemplary embodiment, R₅ is —NEₐE_{b}, and Eₐ and E_{b} are independently hydrogen, hydrocarbyl, substituted hydrocarbyl or heterocyclo.

In each of the foregoing embodiments in which the 1,1-cyclopropane(nitrile)(electron-acceptor) corresponds to Formula CP, CP-1, CP-2, CP-3, CP-4, or CP-5 it is generally preferred that the enanatiomeric excess of the stereoisomer over its enantiomer be at least 60%. More preferably, the cyclopropanenitrile(electron-acceptor) corresponds to Formula CP, CP-1, CP-2, CP-3, CP-4, or CP-5 and the enanatiomeric excess of the stereoisomer over its enantiomer is at least 80%. Still more preferably, the cyclopropanenitrile(electron-acceptor) corresponds to Formula CP, CP-1, CP-2, CP-3, CP-4, or CP-5 and the enanatiomeric excess of the stereoisomer over its enantiomer is at least 90%. In certain embodiments, the cyclopropanenitrile(electron-acceptor) corresponds to Formula CP, CP-1, CP-2, CP-3, CP-4, or CP-5, and the enanatiomeric excess of the stereoisomer over its enantiomer is at least 95%.

In any of the foregoing embodiments in which the cyclopropane(nitrile)(electron-acceptor) corresponds to Formula CP, CP-1, CP-2, CP-3, CP-4, or CP-5, it is generally preferred that the ratio of the stereoisomer to its diastereomer, it is generally preferred that the stereoisomer to diasteromer ratio (i.e., the trans:cis ratio) be greater than 60:1, more preferably greater than 80:1. In certain embodiments in which the cyclopropanenitrile(electron-acceptor) corresponds to Formula CP, CP-1, CP-2, CP-3, CP-4, or CP-5, it is generally preferred that the ratio of the stereoisomer to its diastereomer be greater than 90:1, more preferably greater than 98:1, still more preferably at least 99:1 and still more preferably at least 99:1, respectively.

Cyclopropanation

As summarized in Table 1, using the typical one-pot protocol that has been enjoyed by Co(11)-based MRC, styrene could be effectively cyclopropanated by 1 mol % of [Co(P1)] in dichloromethane with ethyl α-cyanodiazoacetate (ECDA) at room temperature, affording the desired product in almost quantitative yield with promising diastereo- and enantioselectivity (entry 1). To further improve the stereoselectivities, different solvents were evaluated. When the reaction was carried out in chlorobenzene, it increased the enantiomeric excess with no change in the diastereomeric ratio (entry 2). Further improvement in enantioselectivity was observed when dichloroethane was used as the solvent, but with a decreased diastereoselectivity (entry 3). To diverge from using chlorinated solvents, the reaction was then tested in toluene, resulting in improved diastereo- and enantio-selective controls (entry 4). Subsequent experiments indicated n-hexane is the solvent of choice for the catalytic reaction. It provided the best enantiomeric excess and diastereomeric ratio while maintaining an excellent yield (entry 5). Under the same reaction conditions, use of tert-butyl α-cyanodiazoacetate (t-BCDA) instead of ECDA afforded the corresponding E-cyclopropane as the only diastereomer in 91% ee, albeit in a relatively lower yield (entry 6). The enantioselectivity was further enhanced to 95% ee without affecting the excellent diastereoselectivity when the reaction was executed at 0° C. (entry 7). A continued increase in enantiocontrol was observed when the reaction temperature was further lowered to −20° C., achieving 98% ee and with the preservation of the complete E-diastereoselectivity (entry 8). To our delight, the reaction yield surprisingly rose back to 96%, presumably due to elimination of possible side reactions associated with n-hexane at this low temperature.

TABLE 1
Asymmetric Cyclopropanation of Styrene with α-Cyano Diazoacetate
by D₂-Symmetric Chiral Cobalt(II) Porphyrin [Co(P1)].^{a}

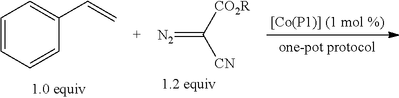

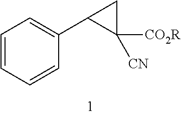

| entry | R | solvent | temp (° C.) | yield (%)^{b} | E:Z^{c} | ee (%)^{d} |
|---|---|---|---|---|---|---|
| 1 | Et | CH₂Cl₂ | 25 | 99 | 84:16 | 62 |
| 2 | Et | C₆H₅Cl | 25 | 92 | 84:16 | 66 |
| 3 | Et | C₂H₄Cl₂ | 25 | 99 | 81:19 | 71 |
| 4 | Et | C₆H₅Me | 25 | 94 | 85:15 | 70 |
| 5 | Et | n-C₆H₁₄ | 25 | 99 | 88:12 | 74 |
| 6 | t-Bu | n-C₆H₁₄ | 25 | 89 | >99:1 | 91 |
| 7 | t-Bu | n-C₆H₁₄ | 0 | 83 | >99:1 | 95 |
| 8 | t-Bu | n-C₆H₁₄ | −20 | 96 | >99:1 | 98 |

^{a}Performed in one-pot fashion for 24 h using 1 mol % [Co(P1)] under N₂ with 1.0 equiv of styrene and 1.2 equiv of α-cyano diazoacetates. [styrene] = 0.25M.
^{b}Isolated yields.
^{c}Determined by NMR.
^{d}Enantiomeric excess of E major diastereomer determined by chiral HPLC.

With the success of asymmetric cyclopropanation of styrene, the scope of the [Co(P1)]/t-BCDA-based catalytic system was then investigated in detail. As summarized in the top part of Table 2, styrene derivatives bearing substituents with varied electronic properties could also be successfully cyclopropanated under similar reaction conditions. For example, the cyclopropanation of styrene derivatives substituted with electron-donating MeO— as well as electron-withdrawing CF₃- and NO₂-groups productively generated the corresponding cyclopropanenitrileesters 1b-d with essentially the same high stereoselectivities as the styrene product 1a, even though in relatively lower yields (entries 1-4). Furthermore, even the extremely electron-poor pentafluorostyrene could be cyclopropanated by [Co(P1)], affording the desired product 1e with essentially complete control of both diastereo- and enantioselectivity, albeit in a lower yield (entry 5). The absolute configuration of 1e was established as [1R,2S] by X-ray crystal structural analysis (see the Examples).

TABLE 2
[Co(P1)]-Catalyzed Diastereo- and Enantioselective Cyclopropanation of
Alkenes with t-Butyl α-Cyanodiazoacetate.^{a}

| | | | | | |
|---|---|---|---|---|---|
| entry | cyclopropane | yield (%)^{b} | E:Z^{c} | ee (%)^{d} | [α]^{e} |

electron-rich and -poor aromatic olefins

| | | | | | | |
|---|---|---|---|---|---|---|
| 1 | 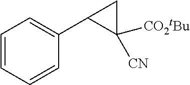 | 1a | 96 | >99:1 | 98 | (−) |
| 2^{g} | 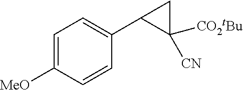 | 1b | 88 | >99:1 | 99 | (−) |
| 3^{g} | 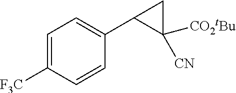 | 1c | 81 | >99:1 | 98 | (−) |
| 4^{g} | 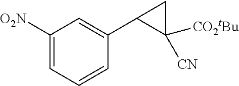 | 1d | 90 | >99:1 | 98 | (−) |
| 5^{h} | 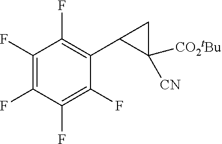 | 1e | 73 | >99:1 | 99 | (−)^{f} |

electron-deficient non-aromatic olefins

| | | | | | | |
|---|---|---|---|---|---|---|
| 6ⁱ | 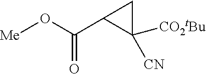 | 1f | 90 | >99:1 | 88 | (−) |
| 7ⁱ | 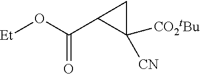 | 1g | 79 | >99:1 | 92 | (−) |
| 8^{j} | 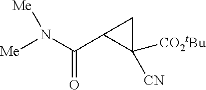 | 1h | 99 | >99:1 | 87 | (−) |
| 9^{k} | 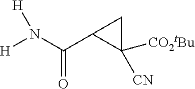 | 1i | 72 | >99:1 | 82 | (−) |
| 10^{j} | 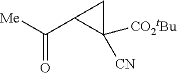 | 1j | 81 | >99:1 | 92 | (−) |
| 11^{j} | 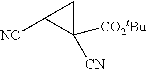 | 1k | 99 | 72:28 | 82 | (−) |

simple aliphatic and electron-rich olefins

| | | | | | | |
|---|---|---|---|---|---|---|
| 12^{l} | 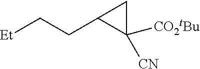 | 1l | 86 | >99:1 | 96^{o} | (−) |
| 13^{l} | 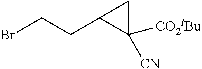 | 1m | 72 | >99:1 | 92^{o} | (−) |
| 14^{l} | 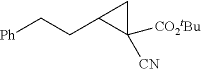 | 1n | 90 | >99:1 | 91 | (−) |
| 16^{m} | 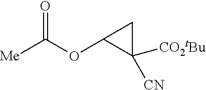 | 1o | 97 | >99:1 | 71 | (+) |
| 17^{l} | 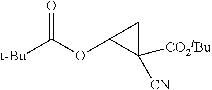 | 1p | 86 | >99:1 | 82 | (+) |
| 18^{l} | 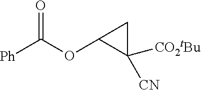 | 1q | 80 | >99:1 | 88 | (−) |

^{a}Performed in n-hexane at −20° C. for 24 h using 1 mol % [Co(P1)] under N₂ with 1.0 equiv of alkene and 1.2 equiv of t-BCDA. [alkene] = 0.25 M.
^{b}Isolated yields.
^{c}Determined by NMR.
^{d}ee of E isomer determined by chiral HPLC.
^{e}Sign of optical rotation.
^{f}[1R,2S] absolute configuration determined by X-ray crystal structural analysis and optical rotation.
^{g}0° C. → RT.
^{h}0° C. → RT; 5 mol %.
ⁱIn C₂H₄Cl₂; alkene:t-BCDA = 5:1; 5 mol %.
^{j}In C₂H₄Cl₂; alkene:t-BCDA = 1:2.5; 5 mol %.
^{k}In C₂H₄Cl₂ at RT; alkene:t-BCDA = 1:2.5; 5 mol %.
^{l}No solvent; 48 h; 5 mol %.
^{m}At RT; no solvent; 48 h; 5 mol %.
^{o}ee of E isomer determined by chiral HPLC via derivatization.

In addition to aromatic olefins, [Co(P1)] was also shown to be an effective catalyst for the cyclopropanation of electron-deficient olefins with CDA, another unique catalytic property of [Co(Por)]-based MRC that is absent in existing non-metalloradical-based catalytic systems. As presented in the middle part of Table 2, various α,β-unsaturated carbonyl compounds and nitriles could be selectively cyclopropanated with t-BCDA by [Co(P1)], furnishing a series of densely functionalized cyclopropane structures. For instance, under modified reaction conditions (Table S1 in the Examples), both methyl and ethyl acrylates could be catalytically converted to the desired cyclopropanenitrilediesters 1f and 1g in 90% and 79% yields, respectively, as single diastereomers with high enantiocontrol (entries 6 and 7). Both substituted and primary acrylamides were also suitable substrates for the catalytic system, providing the corresponding cyclopropane derivatives 1h and 1i bearing three different electron-withdrawing functionalities, including cyano, amido and ester groups, in similar high yields and stereoselectivities (entries 8 and 9). It is notable that all the functional groups were well tolerated; potentially competitive N—H carbene insertion was not observed. Similarly, cyclopropane structures containing ketone, amido and ester groups as three different ring-substituents could be stereoselectively constructed from the reactions of acryl ketones as demonstrated by the formation of 1j (entry 10). Electron-deficient alkenes bearing cyano groups, such as acrylonitriles, could also be successfully cyclopropanated, as exemplified with the nearly quantitative formation of 1k, albeit in lower stereoselectivities (entry 11).

Besides electron-deficient non-aromatic olefins, other non-aromatic olefins were also found to be suitable substrates for the [Co(P1)]/t-BCDA-based catalytic system. As displayed in the bottom part of Table 2, simple aliphatic olefins such as 1-hexene, 1-octene and 4-phenyl-1-butene could be fruitfully converted to the desired products in high yields as single diastereomers with high enantioselectivities when the cyclopropanation reactions were conducted under solvent-free conditions (entries 12-14). Under similar conditions, electron-rich vinyl esters such as vinyl acetate, pivalate, and benzoate could also be productively cyclopropanated, offering the corresponding donor-acceptor cyclopropanes in high yields as sole diastereomers, albeit in relatively low enantiomeric excesses (entries 16-18).

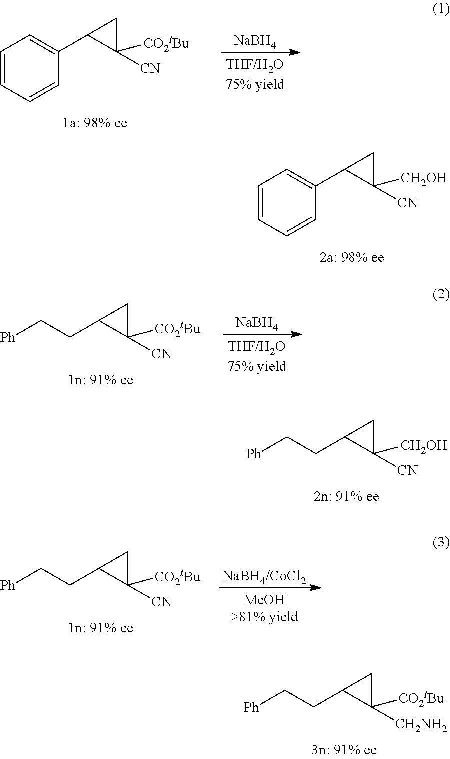

With a viable route to these highly functionalized cyclopropyl nitrileesters in enantioenriched forms by ([Co(P1)]-catalyzed cyclopropanation, we are currently working on exploring their potential applications as chiral building blocks for various stereoselective synthesis (Scheme A1). As initial part of this exploration, the ester groups in both 1a and 1n could be selectively reduced to the corresponding primary alcohols 2a and 2n with full retention of the configuration (eqs 1 and 2). Alternatively, the cyano group in 1n was discriminately converted to a primary amine without affecting the ester functionality under different reduction conditions (eq 3). Again, the stereochemistry was completely preserved.

In one embodiment, the ester group of a 1,1-cyclopropanenitrileester corresponding to Formula CP-1, CP-3 or CP-5 is selectively reduced to the corresponding alcohol as illustrated in the following reaction schemes using a reducing agent such as CoCl₂:

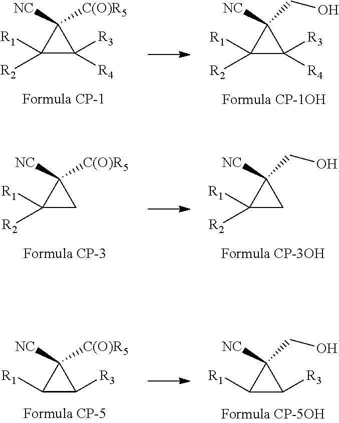

wherein R₁, R₂, R₃, R₄ and R₅ are as defined in connection with Formula CP-1, CP-3 and CP-5, respectively. In addition, stereoselectivities are preserved, thus producing stereoisomers of the alcohols corresponding to Formula CP-1OH, CP-3OH, and CP-5OH, respectively, having an enantiomeric excess over other isomers and a ratio to any diastereomers are previously described in connection with the 1,1-cyclopropane-nitrile(electron-acceptor) corresponding to Formula CP-1, CP-3 or CP-5.

In one embodiment, the cyano group of a 1,1-cyclopropanenitrile-ester corresponding to Formula CP-1, CP-3 or CP-5 is selectively reduced to the corresponding amine as illustrated in the following reaction schemes using a reducing agent such as NaBH₄ and CoCl₂:

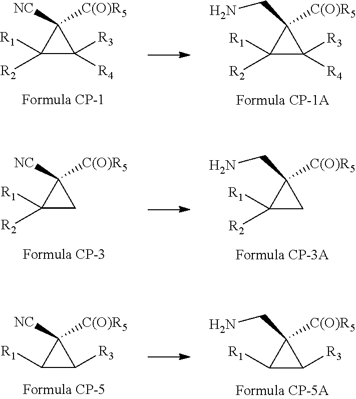

wherein R₁, R₂, R₃, R₄ and R₅ are as defined in connection with Formula CP-1, CP-3 and CP-5, respectively. In addition, stereoselectivities are preserved, thus producing stereoisomers of the amines corresponding to Formula CP-1A, CP-3A, and CP-5A, respectively, having an enantiomeric excess over other isomers and a ratio to any diastereomers are previously described in connection with the 1,1-cyclopropane-nitrile(electron-acceptor) corresponding to Formula CP-1, CP-3 or CP-5.

Similarly, in one embodiment the cyano group of a 1,1-cyclopropanenitrileester corresponding to Formula CP-1, CP-3 or CP-5 may be selectively converted to produce the corresponding 1,1-cyclopropanediester. In another embodiment, the cyano group of a 1,1-cyclopropanenitrileester is converted to an amine (as illustrated above in connection with Formulae CP-1A, CP-3A, and CP-5A, respectively) and the ester group of the same starting material (i.e., the 1,1-cyclopropanenitrileester) is converted to the corresponding alcohol (as illustrated in connection with Formulae CP-1OH, CP-3OH, and CP-5OH) to produce a 1,1-cyclopropyl-aminoalcohol. In another embodiment, the cyano group of a 1,1-cyclopropanenitrileester is converted to an amine (as illustrated above in connection with Formulae CP-1A, CP-3A, and CP-5A, respectively) and the ester group of the same starting material (i.e., the 1,1-cyclopropanenitrileester) is converted to the corresponding carboxylic acid to produce a 1,1-cyclopropyl-aminoacid. In another embodiment, the cyano group of a 1,1-cyclopropanenitrileester is converted to an amide to produce a 1,1-cyclopropyl-amideester.

In summary, we have demonstrated that [Co(P1)] is a versatile and efficient catalyst for highly diastereo- and enantio-selective cyclopropanation of a broad range of different alkenes with α-cyanodiazoacetates. Preliminary results showed some activated internal olefins can also be applied for the [Co(P1)]/t-BCDA-based catalytic system. For example, norbornene could be cyclopropanated to afford the desired product in 66% yield in favor of exo-isomer (exo:endo=83:17). See Examples for its X-ray structural determination and other characterization data. The Co(II)-based system represents the first successful example of using this class of acceptor/acceptor-substituted diazo reagents for the asymmetric cyclopropanation process. The resulting chiral nonracemic cyclopropane derivatives bearing densely functionalized groups possess a myriad of potential synthetic and biological applications (Scheme 1). More broadly, the establishment of α-cyanodiazoacetates as effective and selective carbene sources for cyclopropanation, taken together with other recent successes in the area, may encourage further development of new catalytic systems for the wide use of this and other acceptor/acceptor-substituted diazo reagents for various stereoselective carbene transfer processes.

Having described the invention in detail, it will be apparent that modifications and variations are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

TABLE S1
Asymmetric Cyclopropanation of Methyl Acrylate with α-Cyano
Diazoacetate by D₂-Symmetric Chiral Cobalt(II) Porphyrin [Co(P1)].

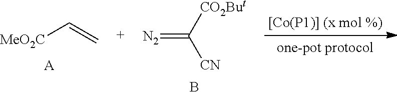

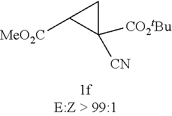

| | | | Cat. | Temp. | Yield | Ee |
|---|---|---|---|---|---|---|
| Entry | A:B | Solvent | Loading | (° C.) | (%)^{a} | (%)^{b} |
| 1 | 1:5 | n-Hexane | 2 mol % | 25 | 99 | 80 |
| 2 | 1:5 | Dichloromethane | 2 mol % | 25 | 98 | 76 |
| 3 | 1:5 | 1,2-Dichlorobenzene | 2 mol % | 25 | 95 | 82 |
| 4 | 1:5 | Toluene | 2 mol % | 25 | 88 | 80 |
| 5 | 1:5 | Chlorobenzene | 2 mol % | 25 | 85 | 77 |
| 6 | 1:5 | 1,2-Dichloroethane | 2 mol % | 25 | 88 | 83 |
| 7 | 1:5 | 1,2-Dichloroethane | 2 mol % | −20 | 75 | 88 |
| 8 | 1:5 | 1,2-Dichloroethane | 5 mol % | −20 | 90 | 88 |
| 9 | 1.2:1 | 1,2-Dichloroethane | 2 mol % | 25 | 76 | 82 |
| 10 | 1.2:1 | 1,2-Dichloroethane | 5 mol % | 25 | 74 | 82 |

^{a}Isolated yields.
^{b}Enantiomeric excess of E major diastereomer determined by chiral HPLC.

General Considerations. All reactions were carried out under a nitrogen atmosphere in oven-dried glassware following standard Schlenk techniques. Hexane (Reagent Plus, ≧99%) was used directly from Sigma-Aldrich Chemical Co. Chlorobenzene was distilled under nitrogen from calcium hydride. α-Nitro-diazoacetates were synthesized following the reported procedure¹. Thin layer chromatography was performed on Merck TLC plates (silica gel 60 F254). Flash column chromatography was performed with ICN silica gel (60 Å, 230-400 mesh, 32-63 μm). Proton and carbon nuclear magnetic resonance spectra (¹H NMR and ¹³C NMR) were recorded on a Bruker 250-MHz instrument and referenced with respect to internal TMS standard. HPLC measurements were carried out on a Shimadzu HPLC system with Whelk-O1, Chiralcel OD-H, OJ-H, and AD-H columns. Infared spectra were measured with a Nicolet Avatar 320 spectrometer with a Smart Miracle accessory. HRMS data was obtained on an Agilent 1100 LC/MS ESI/TOF mass spectrometer with electrospray ionization.

Preparation of TfN₃ hexane solution:¹ A solution of sodium azide (10 g, 150 mmol) and tetrabutylammonium hydrogen sulfate, Bu₄NHSO₄, (0.56 g, 1.64 mmol) in distilled water (30 mL) was cooled to 0° C. A solution of triflic anhydride (8.46 g, 5.0 mL, 30 mmol) in hexane (25 mL) was then slowly added, and the resulting clear solution was stirred for an additional 1 h at 0° C. The reaction mixture was then extracted with hexane (25 mL), and the organic layer was dried over sodium hydroxide pellets and decanted. The hexane solution of triflyl azide was used immediately in the subsequent reaction. Alternatively, it could be stored at −15° C. for several weeks without significant decomposition. The concentration of the azide was estimated based on the total volume of the solution and assuming a quantitative conversion based on the amount of triflic anhydride used.

Typical Procedure for the Preparation of the α-Diazo-cyano Acetate:^{1c} t-Butyl Cyanodiazoacetate. To a stirred solution of the t-butyl cyanoacetate (3.74 g, 20 mmol) in acetonitrile (15 mL) under N₂ was added the above triflyl azide solution (50 mL, 30 mmol) in hexane. Pyridine (4 mL, 53 mmol) was then added dropwise (over ca. 5 min). The reaction mixture was stirred at room temperature for 24 h, after which the solvent was blowed away by the air flow (Note: DON'T USE ROTARY EVAPORATOR AS IT MAY CAUSE POTENTIAL EXPLOSION). Purification of the crude residue by flash chromatography on silica gel (CHCl₃) afforded the pure diazo ester as a yellow oil (1.7 g, 53%): ¹H NMR (250 MHz, CDCl₃) δ 1.55 (s, 9H); ¹³C NMR (62.5 MHz, CDCl3) δ 159.0, 106.7, 83.7, 50.1, 26.7.

General Procedures for Cyclopropanation of Styrene Derivatives. Catalyst (1 mol %) was placed in an oven-dried, resealable Schlenk tube. The tube was capped with a Teflon screwcap, evacuated, and backfilled with nitrogen. The screwcap was replaced with a rubber septum, and 1.0 equivalent of styrene (0.25 mmol) in 1.0 mL Hexane was added via syringe, after cooled to −20° C., followed by 1.2 equivalents of diazo compound (0.30 mmol). The tube was purged with nitrogen for 1 min and its contents were stirred at room temperature. After the reaction finished, the resulting mixture was concentrated and the residue was purified by flash silica gel chromatography to give the product.

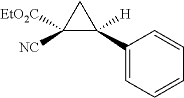

trans-Ethyl 1-cyano-2-phenylcyclopropanecarboxylate: [α]²⁰_{D}=−124.0 (c=1.17, CHCl₃). Trans-: ¹H NMR (250 MHz, CDC1₃): δ 7.30-7.19 (m, 5H), 4.28-4.19 (m, 2H), 3.13-3.06 (m, 1H), 2.12-1.92 (m, 2H), 1.29 (t, J=7.25 Hz, 3H). ¹³C NMR (62.5 MHz, CDC1₃): δ 167.4, 133.0, 128.8, 128.3, 116.4, 63.1, 35.4, 22.8, 19.9, 14.1.

Cis-: ¹H NMR (250 MHz, CDC1₃): δ 7.30-7.19 (m, 5H), 3.92-3.88 (m, 2H), 3.26-3.18 (m, 1H), 2.32-2.27 (m, 1H), 1.95-1.92 (m, 1H), 0.95 (t, J=7.25 Hz, 3H). ¹³C NMR (62.5 MHz, CDC1₃): δ 164.2, 132.1, 129.3, 128.6, 119.1, 62.5, 36.4, 23.0, 21.0, 13.9.

HRMS (ESI) ([M+H]⁺) Calcd. for C13H14NO2: 216.1025. Found 216.1015

HPLC analysis: ee(trans)=74%. OJ-H (99% hexanes: 1% isopropanol, 0.8 mL/min) trans-isomer: tₘₐⱼₒᵣ=78.6 min, tₘᵢₙₒᵣ=106.4 min. ee(cis)=23%. OJ-H (99% hexanes: 1% isopropanol, 0.8 mL/min) trans-isomer: tₘₐⱼₒᵣ=47.3 min, tₘᵢₙₒᵣ=151.0 min.

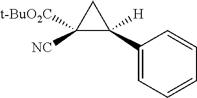

trans-t-Butyl 1-cyano-2-phenylcyclopropanecarboxylate: [α]²⁰_{D}=−156.5 (c=1.03, CHCl₃). Trans-: ¹H NMR (250 MHz, CDC1₃): δ 7.30-7.19 (m, 5H), 3.04-2.97 (m, 1H), 2.03-1.91 (m, 2H), 1.46 (s, 9H). ¹³C NMR (62.5 MHz, CDC1₃): δ 166.1, 133.3, 128.7, 128.4, 128.3, 116.7, 84.2, 34.8, 28.0, 23.7, 22.5.

HRMS (ESI) ([M+H]⁺) Calcd. for C15H18NO2: 244.1338. Found 244.1328

HPLC analysis: ee(trans)=98%. OJ-H (98.5% hexanes: 1.5% isopropanol, 0.8 mL/min) trans-isomer: tₘₐⱼₒᵣ=tₘᵢₙₒᵣ=17.0 min, 20.3 min.

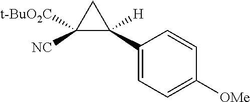

trans-t-Butyl 1-cyano-2-(4-methoxyphenyl)cyclopropane-carboxylate: [α]²⁰_{D}=−155.6 (c=1.65, CHCl₃). Trans-: ¹H NMR (250 MHz, CDC1₃): δ 7.13 (d, J=8.75, 2H), 6.82 (d, J=8.75, 2H), 3.72 (s, 3H), 3.00-2.93 (m, 1H), 2.01-1.86 (m, 2H), 1.45 (s, 9H). ¹³C NMR (62.5 MHz, CDC1₃): δ 166.2, 159.6, 129.5, 125.2, 116.9, 114.2, 84.0, 55.3, 34.6, 28.0, 23.7, 22.6.

HRMS (ESI) ([M+H]⁺) Calcd. for C16H20NO3: 274.1443. Found 274.1432

HPLC analysis: ee(trans)=98.6%. OJ-H (98.5% hexanes: 1.5% isopropanol, 0.8 mL/min) trans-isomer: tₘₐⱼₒᵣ=36.6 min, tₘᵢₙₒᵣ=40.4 min.

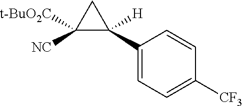

t-Butyl 1-cyano-2-(4-methoxyphenyl)cyclopropanecar-boxylate: [α]²⁰_{D}=−155.6 (c=1.65, CHCl₃). Trans-: ¹H NMR (250 MHz, CDC1₃): δ 7.56 (d, J=8.25, 2H), 7.33 (d, J=8.25, 2H), 3.08-3.01 (m, 1H), 2.09-2.03 (m, 2H), 1.47 (s, 9H). ¹³C NMR (62.5 MHz, CDC1₃): δ 165.6, 137.4, 130.5, 128.8, 125.7, 116.3, 84.6, 33.9, 27.9, 23.8, 22.5.

HRMS (ESI) ([M+H]⁺) Calcd. for C16H17F3NO2: 312.1211. Found 312.1209

HPLC analysis: ee(trans)=98%. Whelk-O1 (99% hexanes: 1.0% isopropanol, 1.0 mL/min) trans-isomer: tₘₐⱼₒᵣ=21.3 min, tₘᵢₙₒᵣ=28.5 min.

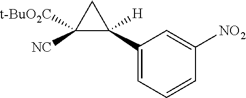

trans-t-Butyl 1-cyano-2-(3-nitrophenyl)cyclopropanecarboxylate: [α]²⁰_{D}=−126.2 (c=1.84, CHCl₃). Trans-: ¹H NMR (250 MHz, CDC1₃): δ 8.13 (m, 2H), 7.52 (m, 2H), 3.15-3.08 (m, 1H), 2.3-1.99 (m, 2H), 1.48 (s, 9H). ¹³C NMR (62.5 MHz, CDC1₃): δ 165.3, 148.4, 135.6, 134.2, 129.9, 123.8, 123.4, 116.1, 84.9, 33.4, 27.9, 23.8, 22.4.

HRMS (ESI) ([M+H]⁺) Calcd. for C15H17N2O4: 289.1188. Found 289.1181

HPLC analysis: ee(trans)=98%. OJ-H (98.5% hexanes: 1.5% isopropanol, 0.8 mL/min) trans-isomer: tₘₐⱼₒᵣ=137.3 min, tₘᵢₙₒᵣ=77.6 min.

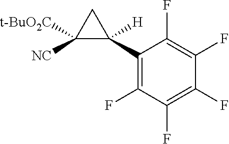

trans-t-Butyl 1-cyano-2-(perfluorophenyl)cyclopropanecarboxylate: [α]²⁰_{D}=−23.2 (c=0.70, CHCl₃). Trans-: ¹H NMR (250 MHz, CDC1₃): δ 2.82-2.75 (m, 1H), 2.18-2.07 (m, 2H), 1.48 (s, 9H). ¹³C NMR (62.5 MHz, CDC1₃): δ 165.1, 116.0, 85.1, 27.9, 23.4, 22.6, 21.7.

HRMS (ESI) ([M+H]⁺) Calcd. for C15H12F5NO2: 334.0866. Found 334.0863

HPLC analysis: ee(trans)=>99%. Whelk-O1 (99% hexanes: 1.0% isopropanol, 1.0 mL/min) trans-isomer: tₘₐⱼₒᵣ=8.0 min, tₘᵢₙₒᵣ=9.3 min.

X-Ray data for t-Butyl 1-cyano-2-(perfluorophenyl)cyclo-propanecarboxylate: The X-ray intensities were measured using Bruker-AXS SMART APEX/CCD diffractometer (CuKa, λ=1.54178 Å). Indexing was performed using SMART v5.625. Frames were integrated with SaintPlus 6.01 software package. Absorption correction was performed by multi-scan method implemented in SADABS. The structure was solved using SHELXS-97 and refined using SHELXL-97 contained in SHELXTL v6.10 and WinGX v1.70.01 programs packages. All non-hydrogen atoms were refined anisotropically. Absolute configuration (and absolute structure) was established by anomalous-dispersion effects in diffraction measurements on the crystal. Crystal data and refinement conditions are shown in Table S2.

TABLE S2
Crystal data and structure refinement

| | |
|---|---|
| Identification code | p212121 |
| Empirical formula | C15 H12 F5 N O2 |
| Formula weight | 333.26 |
| Temperature | 100(2) K. |
| Wavelength | 1.54178 Å |
| Crystal system | Orthorhombic |
| Space group | P2(1)2(1)2(1) |
| Unit cell dimensions | a = 5.97100(10) Å = 90°. |
| | b = 9.7986(2) Å = 90°. |
| | c = 24.7657(6) Å = 90°. |
| Volume | 1448.98(5) Å³ |
| Z | 4 |
| Density (calculated) | 1.528 Mg/m³ |
| Absorption coefficient | 1.277 mm⁻¹ |
| F(000)680 | |
| Crystal size | 0.40 × 0.35 × 0.35 mm³ |
| Theta range for data collection | 3.57 to 68.24°. |
| Index ranges | −6 <= h <= 7, −11 <= k <= 11, |
| | −28 <= l <= 28 |
| Reflections collected | 12553 |
| Independent reflections | 2580 [R(int) = 0.0292] |
| Completeness to theta = 68.24° | 98.5% |
| Absorption correction | Semi-empirical from equivalents |
| Max. and min. transmission | 0.6634 and 0.6290 |
| Refinement method | Full-matrix least-squares on F² |
| Data/restraints/parameters | 2580/0/211 |
| Goodness-of-fit on F2 | 1.129 |
| Final R indices [I > 2sigma(I)] | R1 = 0.0239, wR2 = 0.0611 |
| R indices (all data) | R1 = 0.0243, wR2 = 0.0613 |
| Absolute structure parameter | 0.04(9) |
| Largest diff. peak and hole | 0.139 and −0.198 e.Å⁻³ |

General Procedures for Cyclopropanation of Electron Deficient Olefins: Catalyst (5 mol %) was placed in an oven-dried, resealable Schlenk tube. The tube was capped with a Teflon screwcap, evacuated, and backfilled with nitrogen. The screwcap was replaced with a rubber septum, and 1.0 equivalent of alkene (0.10 mmol) in 1.0 mL Hexane was added via syringe, after cooled to −20° C., followed by 2.5 equivalents of diazo compound (0.25 mmol). The tube was purged with nitrogen for 1 min and its contents were stirred at room temperature. After the reaction finished, the resulting mixture was concentrated and the residue was purified by flash silica gel chromatography to furnish the product.

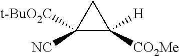

trans-1-t-Butyl 2-methyl 1-cyanocyclopropane-1,2-dicarboxylate: [α]²⁰_{D}=−176.6 (80% ee) (c=0.48, CHCl₃). Trans-: ¹H NMR (250 MHz, CDC1₃): δ 3.75 (s, 3H), 2.57-2.51 (m, 1H), 2.01-1.96 (m, 1H), 1.85-1.80 (m, 1H), 1.45 (s, 9H). ¹³C NMR (62.5 MHz, CDC1₃): δ 167.5, 164.4, 115.1, 85.2, 53.0, 29.7, 27.8, 21.8, 21.8.

HRMS (ESI) ([M+H]⁺) Calcd. for C11H16NO4: 226.1079. Found 226.1074

HPLC analysis: ee(trans)=88%. OJ-H (98.5% hexanes: 1.5% isopropanol, 0.8 mL/min) trans-isomer: tₘₐⱼₒᵣ=18.9 min, tₘᵢₙₒᵣ=22.0 min.

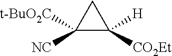

trans-1-t-Butyl 2-ethyl 1-cyanocyclopropane-1,2-dicarboxylate: [α]²⁰_{D}=−126.0 (c=0.29, CHCl₃). Trans-: ¹H NMR (250 MHz, CDC1₃): δ 4.25-4.16 (m, 2H), 2.55-2.48 (m, 1H), 2.00-1.95 (m, 1H), 1.84-1.78 (m, 1H), 1.45 (s, 9H), 1.26 (t, J=7.0 Hz, 3H). ¹³C NMR (62.5 MHz, CDC1₃): δ 167.0, 164.5, 115.1, 85.1, 62.3, 29.9, 27.8, 21.8, 14.1.

HRMS (ESI) ([M+H]⁺) Calcd. for C12H18NO4: 240.1236. Found 240.1229

HPLC analysis: ee(trans)=92%. OJ-H (98.5% hexanes: 1.5% isopropanol, 0.8 mL/min) trans-isomer: tₘₐⱼₒᵣ=14.9 min, tₘᵢₙₒᵣ=17.9 min.

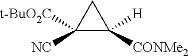

1-t-Butyl 1-cyano-2-(dimethylcarbamoyl)cyclopropanecarboxylate: [α]²⁰_{D}=−52.5 (c=0.15, CHCl₃). Trans-: ¹H NMR (250 MHz, CDC1₃): δ 3.08 (s, 3H), 2.98 (s, 3H), 2.71-2.65 (m, 1H), 2.17-2.12 (m, 1H), 1.77-1.72 (m, 1H), 1.45 (s, 9H). ¹³C NMR (62.5 MHz, CDC1₃): δ 165.3, 164.9, 115.6, 84.8, 37.1, 36.1, 27.8, 21.6, 21.2.

HRMS (ESI) ([M+H]⁺) Calcd. for C12H19N2O3: 239.1396. Found 239.1397

HPLC analysis: ee(trans)=87%. Whelk-O1 (80% hexanes: 20% isopropanol, 1 mL/min) trans-isomer: tₘₐⱼₒᵣ=30.4 min, tₘᵢₙₒᵣ=23.5 min.

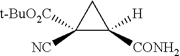

1-t-Butyl 2-carbamoyl-1-cyanocyclopropanecarboxylate: [α]²⁰_{D}=−30.7 (c=0.55, CHCl₃). Trans-: ¹H NMR (250 MHz, CDC1₃): δ 5.73 (s, broad, 2H), 2.43-2.37 (m, 1H), 2.08-2.03 (m, 1H), 1.78-1.72 (m, 1H), 1.45 (s, 9H). ¹³C NMR (62.5 MHz, CDC1₃): δ 167.4, 166.2, 116.2, 84.5, 31.6, 27.9, 21.9, 21.2.

HRMS (ESI) ([M+H]⁺) Calcd. for C10H15N2O3: 211.1083. Found 211.1074

HPLC analysis: ee(trans)=82%. Whelk-O1 (80% hexanes: 20% isopropanol, 1 mL/min) trans-isomer: tₘₐⱼₒᵣ=12.2 min, tₘᵢₙₒᵣ=10.2 min.

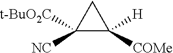

1-t-Butyl 2-acetyl-1-cyanocyclopropanecarboxylate: [α]²⁰_{D}=−179.6 (c=0.15, CHCl₃). Trans-: ¹H NMR (250 MHz, CDC1₃): δ 2.81-2.75 (m, 1H), 2.36 (s, 3H), 2.01-1.96 (m, 1H), 1.77-1.72 (m, 1H), 1.46 (s, 9H). ¹³C NMR (62.5 MHz, CDC1₃): δ 199.5, 164.7, 114.9, 85.1, 35.3, 31.5, 27.8, 23.2, 22.1.

HRMS (ESI) ([M+H]⁺) Calcd. for C11H16NO3: 210.1130. Found 210.1117

HPLC analysis: ee(trans)=92%. OD-H (95% hexanes: 5% isopropanol, 1 mL/min) trans-isomer: tₘₐⱼₒᵣ=tₘᵢₙₒᵣ=12.2 min, 11.3 min.

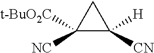

1-t-Butyl 1,2-dicyanocyclopropanecarboxylate: [α]²⁰_{D}=−41.3 (mixture of cis and trans) (c=1.65, CHCl₃). Trans-: ¹H NMR (250 MHz, CDC1₃): δ 2.50-2.43 (m, 1H), 2.02-1.99 (m, 2H), 1.53 (s, 9H). Cis-: ¹H NMR (250 MHz, CDC1₃): δ 2.22-2.17 (m, 1H), 2.02-1.99 (m, 2H), 1.56 (s, 9H). Cis+Trans mixture: ¹³C NMR (62.5 MHz, CDC1₃): δ 162.9, 161.3, 115.8, 115.3, 114.4, 114.1, 86.7, 86.5, 27.8, 27.8, 22.4, 21.5, 20.8, 20.7, 15.0, 14.9. HRMS (ESI) ([M+Na]⁺) Calcd. for C10H12N2NaO2: 215.0796. Found 215.0789. HPLC analysis: ee(trans)=91%. OD-H (95% hexanes: 5% isopropanol, 1 mL/min) trans-isomer: tₘₐⱼₒᵣ=31.6 min, tₘᵢₙₒᵣ=19.9 min.

General Procedures for Cyclopropanation of Aliphatic Alkenes. Catalyst (5 mol %) was placed in an oven-dried, resealable Schlenk tube. The tube was capped with a Teflon screwcap, evacuated, and backfilled with nitrogen. The screwcap was replaced with a rubber septum, and alkene (1.0 mL) was added via syringe, after cooled to −20° C., followed by diazo compound (0.25 mmol). The tube was purged with nitrogen for 1 min and its contents were stirred at room temperature. After the reaction finished, the resulting mixture was concentrated and the residue was purified by flash silica gel chromatography to give the product.

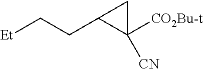

trans-t-Butyl 2-butyl-1-cyanocyclopropanecarboxylate: [α]²⁰_{D}=−10.8 (c=0.24, CHCl₃). Trans-: ¹H NMR (250 MHz, CDC1₃): δ 1.74-1.18 (m, 18H), 0.89-0.83 (m, 3H). ¹³C NMR (62.5 MHz, CDC1₃): δ 166.8, 117.8, 83.6, 30.9, 30.6, 30.0, 27.9, 25.0, 22.3, 20.3, 13.9.

HRMS (ESI) ([M+H]⁺) Calcd. for C13H22NO2: 224.1651. Found 224.1641.

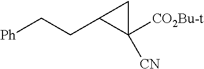

trans-t-Butyl 1-cyano-2-phenethylcyclopropanecarboxylate: [α]²⁰_{D}=−14.3, (c=0.53, CHC13). Trans-: ¹H NMR (250 MHz, CDC13): δ 7.21-7.11 (m, 5H), 2.78-2.70 (m, 2H), 1.89-1.80 (m, 2H), 1.75-1.61 (m, 2H), 1.42 (s, 9H), 1.20-1.15 (m, 1H). ¹³C NMR (62.5 MHz, CDC1₃): δ 166.6, 140.5, 128.6, 128.6, 126.3, 117.7, 83.7, 34.7, 32.2, 30.2, 27.9, 24.8, 20.2.

HRMS (ESI) ([M+Na]⁺) Calcd. for C17H21NNaO2: 294.1470. Found 294.1472

HPLC analysis: ee(trans)=91%. OJ-H (98.5% hexanes: 1.5% isopropanol, 0.8 mL/min) trans-isomer: tₘₐⱼₒᵣ=14.8 min, tₘᵢₙₒᵣ=12.8 min.

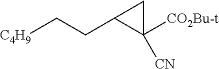

trans-t-Butyl 1-cyano-2-hexylcyclopropanecarboxylate: [α]²⁰_{D}=−12.3 (c=1.07, CHCl₃). Trans-: ¹H NMR (250 MHz, CDC1₃): δ 1.71-1.19 (m, 22H), 0.82 (m, 3H). ¹³C NMR (62.5 MHz, CDC1₃): δ 166.8, 117.8, 83.6, 31.6, 31.0, 30.3, 28.8, 28.5, 27.9, 25.0, 22.6, 20.3, 14.1.

HRMS (ESI) ([M+H]⁺) Calcd. for C15H26NO2: 252.1964. Found 252.1956.

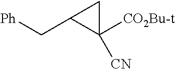

trans-t-Butyl 2-benzyl-1-cyanocyclopropanecarboxylate: [α]²⁰_{D}=−4.8, (c=1.03, CHCl₃). Trans-: ¹H NMR (250 MHz, CDC1₃): δ 7.26-7.18 (m, 5H), 3.07-2.99 (m, 1H), 2.74-2.65 (m, 1H), 2.07-1.99 (m, 1H), 1.76-1.67 (m, 1H), 1.42 (s, 9H), 1.44-1.16 (m, 1H). ¹³C NMR (62.5 MHz, CDC1₃): δ 166.4, 138.5, 128.8, 128.4, 126.9, 117.7, 83.9, 36.0, 31.1, 27.9, 24.7, 20.7.

HRMS (ESI) ([M+H]⁺) Calcd. for C16H20NO2: 258.1494. Found 258.1489.

HPLC analysis: ee(trans)=91%. OJ-H (98.5% hexanes: 1.5% isopropanol, 0.8 mL/min) trans-isomer: tₘₐⱼₒᵣ=18.5 min, tₘᵢₙₒᵣ=16.1 min.

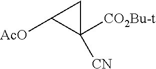

trans-t-Buty 2-acetoxy-1-cyanocyclopropanecarboxylate: [α]²⁰_{D}=+ 73.54, (c=0.091, CHCl₃). Trans-: ¹H NMR (250 MHz, CDC1₃): δ 4.53-4.49 (m, 1H), 2.11 (s, 3H), 1.95-1.79 (m, 2H), 1.44 (s, 9H). ¹³C NMR (62.5 MHz, CDC1₃): δ 170.1, 164.5, 115.2, 84.7, 57.6, 27.9, 22.2, 21.2, 20.5.

HRMS (ESI) ([M+Na]⁺) Calcd. for C11H15NO4Na: 248.0899. Found 248.0881

HPLC analysis: ee(trans)=71%. OD-H (98.5% hexanes: 1.5% isopropanol, 0.8 mL/min) trans-isomer: tₘₐⱼₒᵣ=11.2 min, tₘᵢₙₒᵣ=12.5 min.

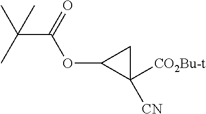

trans-t-Butyl 1-cyano-2-(pivaloyloxy)cyclopropanecar-boxylate: [α]²⁰_{D}=+ 52.88, (c=0.076, CHCl₃). Trans-: ¹H NMR (250 MHz, CDC1₃): δ 4.54-4.43 (m, 1H), 1.94 (t, J=6.70 Hz, 1H), 1.83 (t, J=6.05 Hz, 1H), 1.44 (s, 9H), 1.19 (s, 9H) 1.46 (s, 9H). ¹³C NMR (62.5 MHz, CDC1₃): δ 178.0, 164.6, 115.1, 84.6, 57.5, 38.6, 27.9, 27.0, 22.3, 21.3, HRMS (ESI) ([M+H]⁺) Calcd. for C14H22NO4: 268.1549,

Found 268.1536. HPLC analysis: ee(trans)=82%. OJ-H (98.5% hexanes: 1.5% isopropanol, 0.8 mL/min) trans-isomer: tₘₐⱼₒᵣ=7.5 min, tₘᵢₙₒᵣ=6.6 min.

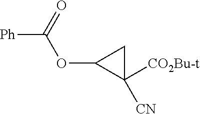

trans-2-(t-Butoxycarbonyl)-2-cyanocyclopropyl benzoate: [α]²⁰_{D}=−23.55, (c=0.064, CHCl₃). Trans-: ¹H NMR (250 MHz, CDC1₃): δ 8.00 (td, J=8.51, 1.65 Hz, 2H), 7.61-7.46 (m, 1H), 7.46-7.31 (m, 2H), 4.81 (dd, J=6.72, 5.44 Hz, 1H), 2.12-1.90 (m, 2H), 1.46 (s, 9H). ¹³C NMR (62.5 MHz, CDC1₃): δ 165.8, 164.5, 134.1, 130.0, 128.7, 128.2, 115.2, 84.8, 58.0, 27.9, 22.7, 21.2, HRMS (ESI) ([M+Na]⁺) Calcd. for C16H17NO4Na: 310.1055. Found 310.1050. HPLC analysis: ee(trans)=88%. OJ-H (98.5% hexanes: 1.5% isopropanol, 0.8 mL/min) trans-isomer: tₘₐⱼₒᵣ=25.3 min, tₘᵢₙₒᵣ=29.6 min.

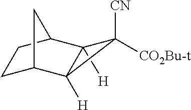

Exo-isomer: ¹H NMR (250 MHz, CDC1₃): δ 2.59-2.58 (m, 2H), 1.71-1.24 (m, 15H), 0.78 (m, 2H). Endo-isomer: ¹H NMR (250 MHz, CDC1₃): δ 2.11-2.03 (m, 2H), 1.71-1.24 (m, 15H), 0.78 (m, 2H). Exo+Endo mixture: ¹³C NMR (62.5 MHz, CDC1₃): δ 166.8, 163.9, 119.6, 118.4, 83.6, 83.4, 36.3, 35.9, 35.2, 31.6, 29.6, 29.4, 28.5, 27.9, 27.6, 20.8, 18.3.

HRMS (ESI) ([M+H]⁺) Calcd. for C14H20NO2: 234.1494. Found 234.1485.

Exo-isomer: The X-ray intensities were measured using Bruker-AXS SMART APEX/CCD diffractometer (CuKa, λ=1.54178 Å). Indexing was performed using SMART v5.625. Frames were integrated with SaintPlus 6.01 software package. Absorption correction was performed by multi-scan method implemented in SADABS. The structure was solved using SHELXS-97 and refined using SHELXL-97 contained in SHELXTL v6.10 and WinGX v1.70.01 programs packages. All non-hydrogen atoms were refined anisotropically. Absolute configuration (and absolute structure) was established by anomalous-dispersion effects in diffraction measurements on the crystal. Crystal data and refinement conditions are shown in Table S3.

TABLE S3
Crystal data and structure refinement

| | |
|---|---|
| Identification code | pnm2(1) |
| Empirical formula | C14 H19 N O2 |
| Formula weight | 233.30 |
| Temperature | 100(2) K. |
| Wavelength | 1.54178 Å |
| Crystal system | Orthorhombic |
| Space group | Pmn2(1) |
| Unit cell dimensions | a = 8.5842(2) Å = 90°. |
| | b = 8.5197(2) Å = 90°. |
| | c = 8.4723(2) Å = 90°. |
| Volume | 619.62(3) Å³ |
| Z | 2 |
| Density (calculated) | 1.250 Mg/m³ |
| Absorption coefficient | 0.662 mm⁻¹ |
| F(000)252 | |
| Crystal size | 0.4 × 0.3 × 0.2 mm³ |
| Theta range for data collection | 5.19 to 68.08°. |
| Index ranges | −9 <= h <= 8, −9 <= k <= 10, |
| | −9 <= l <= 10 |
| Reflections collected | 2596 |
| Independent reflections | 1013 [R(int) = 0.0215] |
| Completeness to theta = 68.08° | 90.7% |
| Absorption correction | SADABS |
| Refinement method | Full-matrix least-squares on F² |
| Data/restraints/parameters | 1013/1/95 |
| Goodness-of-fit on F2 | 1.145 |
| Final R indices [I > 2sigma(I)] | R1 = 0.0375, wR2 = 0.0911 |
| R indices (all data) | R1 = 0.0376, wR2 = 0.0915 |
| Absolute structure parameter | 0.1(3) |
| Largest diff. peak and hole | 0.202 and −0.310 e.Å⁻³ |

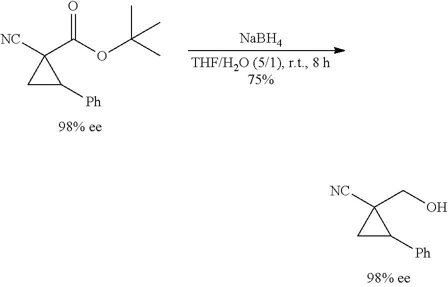

General Procedure for reduction the ester into alcohol:² To a solution of trans-t-Butyl 1-cyano-2-phenylcyclopropanecarboxylate (27 mg, 0.1 mmol) in 1.0 mL of THF:H2O (5:1, 1.0 mL) at ambient temperature was added sodium borohydride (19 mg, 0.5 mmol). Additional NaBH4 (19 mg) was added and stirred overnight and quenched cautiously with 1 N HCl (aq). The mixture was concentrated to remove THF and then partitioned between Et2O/1 N HCl (aq). The organic phase was washed with sat. NaHCO3 (aq) and brine, dried (MgSO4), followed by purification of the residue by column chromatography 13 mg (75%) of 1-(Hydroxymethyl)-2-phenylcyclopropane-carbonitrile as a colorless oil. enantio excess ratio was remained unchanged.

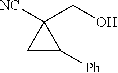

1-(Hydroxymethyl)-2-phenylcyclopropanecarbonitrile: [α]²⁰_{D}=−41.1 (c=0.25, CHCl₃). Trans-: ¹H NMR (250 MHz, CDC1₃): δ 7.30-7.19 (m, 5H), 3.81-3.62 (AB, J1=11.75, J2=34.5, 2H), 2.47-2.41 (m, 1H), 1.91 (s, broad, 1H), 1.70-1.64 (m, 1H), 1.45-1.39 (m, 1H). ¹³C NMR (62.5 MHz, CDCl₃): δ 135.0, 128.6, 128.0, 127.7, 120.0, 65.9, 28.4, 22.5, 17.1.

HRMS (ESI) ([M+H]⁺) Calcd. for C11H12NO: 173.0841. Found 173.0912

HPLC analysis: ee(trans)=98%. OD-H (95% hexanes: 5% isopropanol, 0.8 mL/min) trans-isomer: tₘₐⱼₒᵣ=52.6 min, tₘᵢₙₒᵣ=38.2 min.

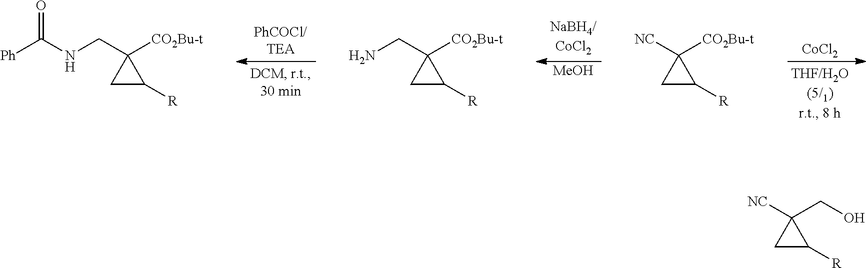

General Procedure for selective reduction of the ester-group or cyano-group using NaBH₄ as the reductant:²

The procedure for reducing the ester group into hydroxyl group is same with described above.

Selectively reduce the cyano group (CN—) into amine-: To a solution of 1-cyano-2-alkyl-cyclopropanecarboxylate (27 mg, 0.1 mmol) and cobaltous chloride (0.2 mmol) in MOH (1.0 mL) was added sodium borohydride (1.0 mmol) in portions. The black precipitate formed was stirred for 1 h at room temperature and was quenched with 2N HCl (aq, 20 mL). After the black precipitate dissolved, the PH value was adjusted to 9 with 2 N NaOH (aq) and extracted with DCM (4×). The organic phase was separated, washed with brine, and dried (Na₂SO₄). Evaporation of solvent gave the crude mixture, which was dissolved in DCM (1.0 ml), followed by addition TEA (30 μL) and PhCOCl (1.2 mmol). After 30 min, remove the solvent and purify the residue by column chromatography furnished the corresponding phenyl carboxylamide as colorless oil.

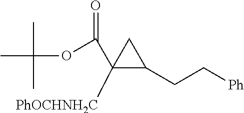

tert-Butyl 1-(benzamidomethyl)-2-phenethylcyclopropanecarboxylate: [α]²⁰_{D}=−2.1 (c=0.58, CHCl₃). Trans-: ¹H NMR (250 MHz, CDC1₃): δ 7.70-7.66 (m, 2H), 7.42-7.33 (m, 3H), 7.19-7.07 (m, 5H), 6.95 (s, br, 1H), 3.66-3.41 (m, 2H), 2.67-2.61 (m, 2H), 1.83-1.73 (m, 2H), 1.49-1.18 (m, 11H), 0.66-0.64 (m, 1H). ¹³C NMR (62.5 MHz, CDC1₃): δ 174.6, 166.6, 141.5, 134.8, 131.4, 128.6, 128.4, 126.9, 125.9, 8.1, 39.2, 36.1, 30.6, 29.4, 28.2, 27.2, 20.3.

HRMS (ESI) ([M+Na]⁺) Calcd. for C24H29NNaO3: 402.2045. Found 402.2040

HPLC analysis: ee(trans)=91%. OJ-H (98.5% hexanes: 1.5% isopropanol, 1.0 mL/min) trans-isomer: tₘₐⱼₒᵣ=36.6 min, tₘᵢₙₒᵣ=18.6 min.

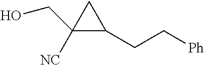

1-(Hydroxymethyl)-2-phenethylcyclopropanecarbonitrile: [α]²⁰_{D}=+ 7.7 (c=0.26, CHCl₃). Trans-: ¹H NMR (250 MHz, CDC1₃): δ 7.23-7.14 (m, 5H), 3.62-3.41 (m, 2H), 2.79-2.71 (m, 2H), 1.88-1.76 (m, 2H), 1.50 (s, broad, 1H), 1.18-0.90 (m, 1H). ¹³C NMR (62.5 MHz, CDC1₃): δ 140.9, 128.6, 128.5, 126.2, 66.1, 34.9, 32.5, 23.5, 19.1, 18.3.

HRMS (ESI) ([M+Na]⁺) Calcd. for C13H15NNaO: 224.1051. Found 224.1042

HPLC analysis: ee(trans)=91%. OD-H (95% hexanes: 5% isopropanol, 1.0 mL/min) trans-isomer: tₘₐⱼₒᵣ=30.1 min, tₘᵢₙₒᵣ=23.5 min.

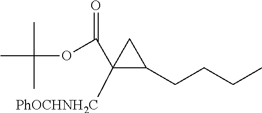

tert-Butyl 1-(benzamidomethyl)-2-butylcyclopropane-carboxylate: [α]²⁰_{D}=−5.5 (c=0.29, CHCl₃). Trans-: ¹H NMR (250 MHz, CDC1₃): δ 7.71-7.68 (m, 2H), 7.46-7.33 (m, 3H), 6.95 (s, br, 1H), 3.71-3.54 (m, 2H), 1.54-1.23 (m, 17H), 0.83-0.76 (m, 3H), 0.68-0.64 (m, 1H). ¹³C NMR (62.5 MHz, CDC1₃): δ 174.8, 166.8, 134.9, 131.3, 128.6, 126.9, 81.0, 39.4, 32.1, 29.2, 28.5, 28.2, 27.7, 22.5, 20.6, 14.1.

HRMS (ESI) ([M+H]⁺) Calcd. for C20H3ONO3: 332.2226. Found 332.2210

HPLC analysis: ee(trans)=96%. Whelk-O1 (98.5% hexanes: 1.5% isopropanol, 1.0 mL/min) trans-isomer: tₘₐⱼₒᵣ=85.3 min, tₘᵢₙₒᵣ=77.7 min.

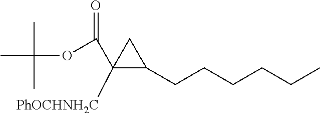

tert-Butyl 1-(benzamidomethyl)-2-hexylcyclopropanecarboxylate: [α]²⁰_{D}=−5.4 (c=0.22, CHCl₃). Trans-: ¹H NMR (250 MHz, CDC13): δ 7.71-7.68 (m, 2H), 7.46-7.34 (m, 3H), 6.94 (s, br, 1H), 3.71-3.54 (m, 2H), 1.52-1.18 (m, 21H), 0.82-0.66 (m, 3H), 0.65 (m, 1H). ¹³C NMR (62.5 MHz, CDC1₃): δ 174.8, 166.8, 134.9, 131.3, 128.6, 126.9, 81.0, 39.4, 31.8, 29.7, 29.2, 29.1, 28.8, 28.2, 27.8, 22.6, 20.6, 14.1.

HRMS (ESI) ([M+H]⁺) Calcd. for C22H34NO3: 360.2539. Found 360.2543

HPLC analysis: ee(trans)=92.6%. Whelk-O1 (98.5% hexanes: 1.5% isopropanol, 1.0 mL/min) trans-isomer: tₘₐⱼₒᵣ=79.8 min, tₘᵢₙₒᵣ=73.1 min.

(a) Cavender, C. J.; Shiner, V. J., Jr. J. Org. Chem. 1972, 37, 3567-3569. (b) Fritschi, S.; Vasella, A. Helv. Chim. Acta 1991, 74, 2024-2034. (c) Charette, A. B.; Wurz, R. P.; Ollevier, T. J. Org. Chem. 2000, 65, 9252-9254.

Schwarz, J. B.; Gibbons, S. E.; Graham, S. R.; Colbry, N. L.; Guzzo, P. R.; Le, V.-D.; Vartanian, M. G.; Kinsora, J. J.; Lotarski, S. M.; Li, Z.; Dickerson, M. R.; Su, T.-Z.; Weber, M. L.; El-Kattan, A.; Thorpe, A. J.; Donevan, S. D.; Taylor, C. P.; Wustrow, D. J. Journal of Medicinal Chemistry 2005, 48, 3026-3035.

What is claimed is:

1. A process for the preparation of a 1,1-cyclopropane(nitrile)(electron-acceptor) comprising treating an olefin with an acceptor/acceptor-substituted α-cyanodiazo reagent in the presence of a catalytic amount of a porphyrin catalyst.

2. The process of claim 1 wherein the porporphyrin catalyst is a D₂-symmetric chiral porphyrin.

3. The process of claim 2 wherein the olefin corresponds to Formula O-1, the diazo reagent corresponds to Formula D-1, and the 1,1-cyclopropane(nitrile)(electron-acceptor) corresponds to Formula CP 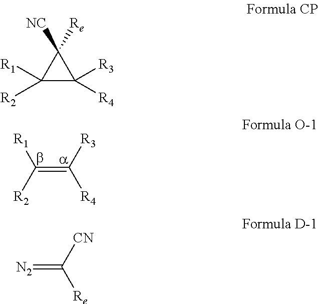
wherein Rₑ is an electron-acceptor and R₁, R₂, R₃, and R₄ are independently hydrogen, hydrocarbyl, substituted hydrocarbyl, heterocyclo, or an electron withdrawing group.

4. The process of claim 1 wherein the olefin corresponds to Formula O-1, the diazo reagent corresponds to Formula D-1, and the 1,1-cyclopropane(nitrile)(electron-acceptor) corresponds to Formula CP 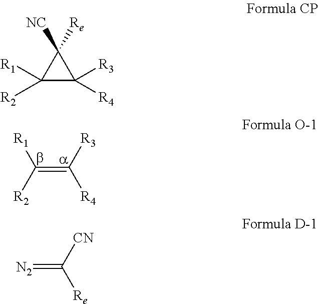
wherein Rₑ is an electron-acceptor and R₁, R₂, R₃, and R₄ are independently hydrogen, hydrocarbyl, substituted hydrocarbyl, heterocyclo, or an electron withdrawing group.

5. The process of claim 4 wherein the diazo reagent corresponds to Formula D-1: 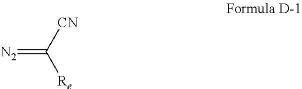
wherein Rₑ is hydroxy, alkoxy, mercapto, halogen, carbonyl, sulfonyl, nitrile, quaternary amine, nitro, trihalomethyl, imine, amidine, oxime, thioketone, thioester, or thioamide.

6. The process of claim 5 wherein Rₑ is hydroxy, alkoxy, mercapto, halogen, carbonyl, sulfonyl, nitrile, quaternary amine, nitro, or trihalomethyl.

7. The process of claim 5 wherein Rₑ is halogen, carbonyl, nitrile, quaternary amine, nitro, or trihalomethyl.

8. The process of claim 5 wherein Rₑ is halogen, carbonyl, nitrile, nitro, or trihalomethyl.

9. The process of claim 5 wherein Rₑ is aldehyde (—C(O)H), ketone (—C(O)R), ester (—C(O)OR), acid (—C(O)OH), acid halide (—C(O)X), amide (—C(O)NRₐR_{b}), or anhydride (—C(O)OC(O)R) where R is hydrocarbyl, substituted hydrocarbyl or heterocyclo, Rₐ and R_{b} are independently hydrogen, hydrocarbyl, substituted hydrocarbyl or heterocyclo, and X is a halogen atom.

10. The process of claim 5 wherein Rₑ is —SO₃H or —SO₂R where R is hydrocarbyl, substituted hydrocarbyl or heterocyclo.

11. The process of claim 5 wherein Rₑ is —N⁺RₐR_{b}R_{c} where Rₐ, R_{b} and R_{c} are independently hydrogen, hydrocarbyl, substituted hydrocarbyl or heterocyclo.

12. The process of claim 4 wherein the olefin corresponds to Formula O-1-EWG: 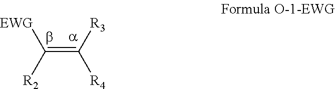
wherein EWG is an electron withdrawing group and R₂, R₃, and R₄ are R₂, R₃, and R₄ are independently hydrogen, hydrocarbyl, substituted hydrocarbyl, heterocyclo, or an electron withdrawing group.

13. The process of claim 4 wherein the olefin corresponds to Formula O-2: 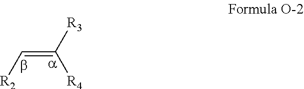
wherein R₂, R₃, and R₄ are independently hydrogen, hydrocarbyl, substituted hydrocarbyl, heterocyclo, or an electron withdrawing group.

14. The process of claim 4 wherein the olefin corresponds to Formula O-3-cis or Formula O-3-trans: 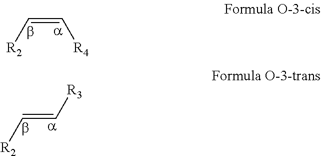
wherein R₂, R₃ and R₄ are independently hydrogen, hydrocarbyl, substituted hydrocarbyl, heterocyclo, or an electron withdrawing group.

15. The process of claim 4 wherein the olefin corresponds to Formula O-4: 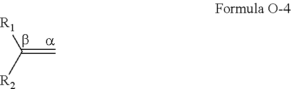
wherein R₁ and R₂ are independently hydrogen, hydrocarbyl, substituted hydrocarbyl, heterocyclo, or an electron withdrawing group.

16. The process of claim 4 wherein the olefin corresponds to Formula O-5: 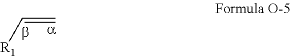
wherein R₁ is hydrogen, hydrocarbyl, substituted hydrocarbyl, heterocyclo, or an electron withdrawing group.

17. The process of claim 4 wherein the porphyrin catalyst is selected from the group consisting of [Co(P1)], [Co(P2)], [Co(P3)], [Co(P4)], [Co(P5)], [Co(P6)] and combinations thereof wherein [Co(P1)], [Co(P2)], [Co(P3)], [Co(P4)], [Co(P5)], and [Co(P6)] have the following Formula:

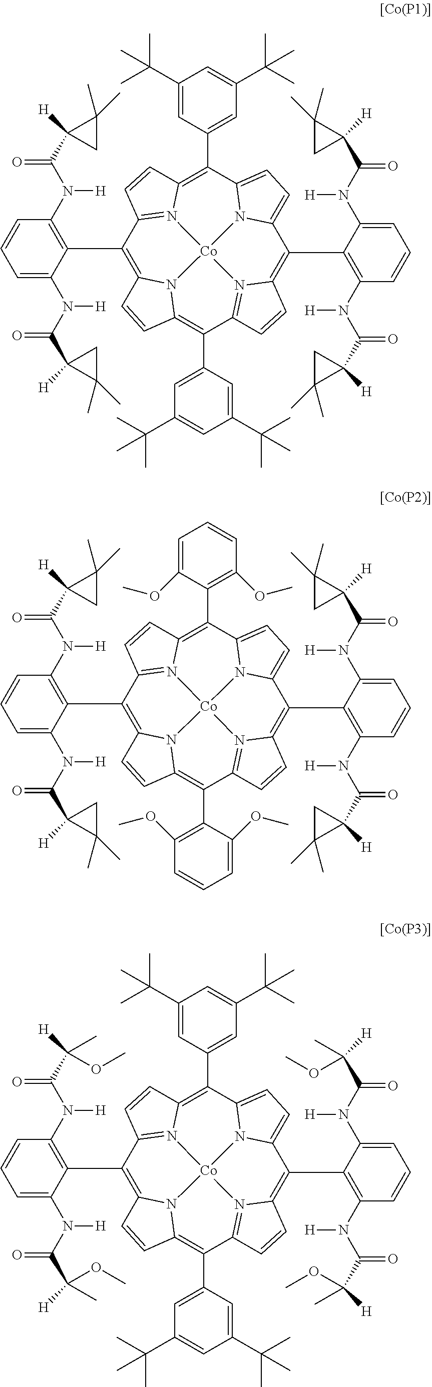

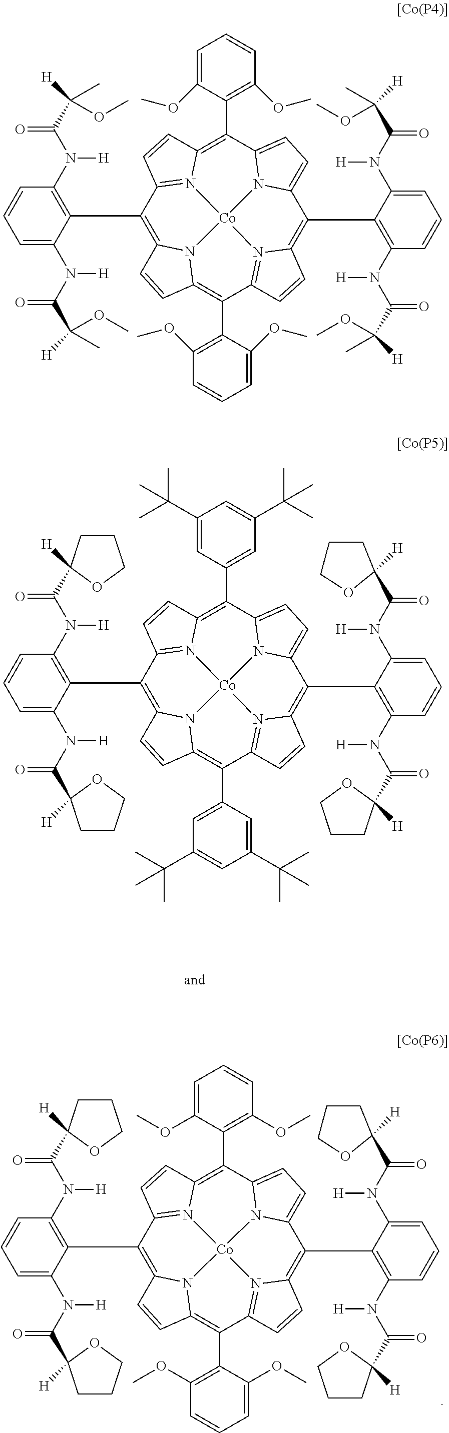

18. The process of claim 1 wherein the porphyrin catalyst is selected from the group consisting of [Co(P1)], [Co(P2)], [Co(P3)], [Co(P4)], [Co(P5)], [Co(P6)] and combinations thereof wherein [Co(P1)], [Co(P2)], [Co(P3)], [Co(P4)], [Co(P5)], and [Co(P6)] have the following Formula:

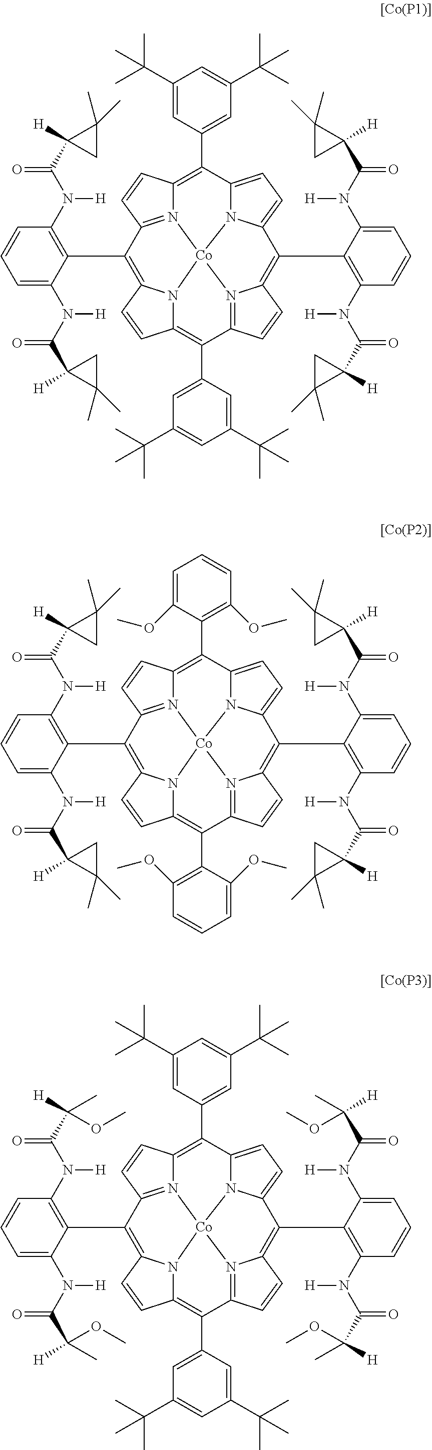

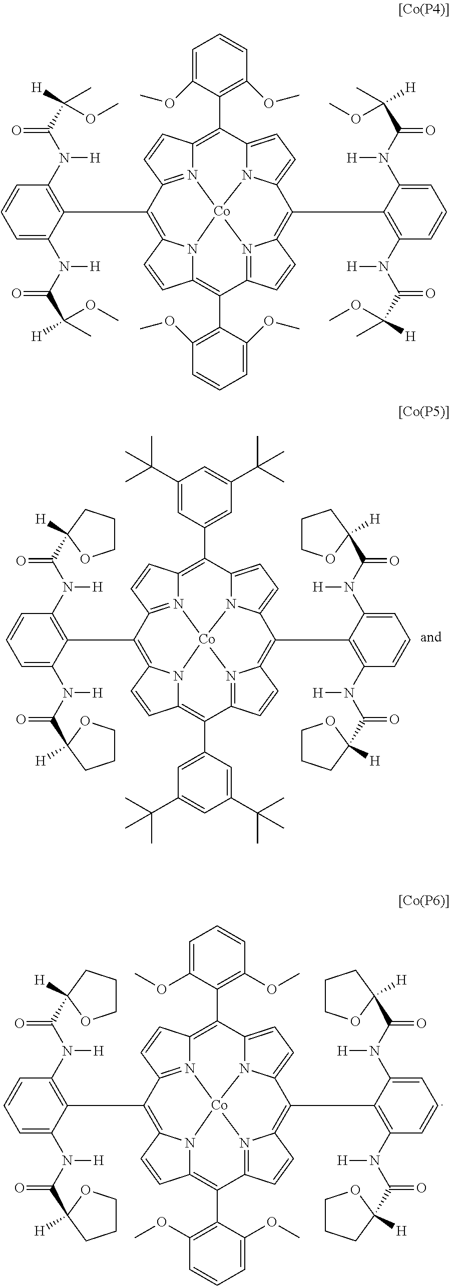

19. A 1,1-cyclopropane(nitrile)(electron-acceptor) corresponding to Formula CP: 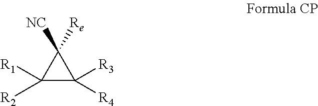
wherein R₁, R₂, R₃, and R₄ are independently hydrogen, hydrocarbyl, substituted hydrocarbyl, heterocyclo, or an electron withdrawing group, and Rₑ is an electron-acceptor.

20. A diazo reagent corresponding to Formula D-1 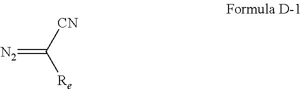
wherein Rₑ is an electron-acceptor.
